(19) United States
(12) Patent Application Publication
(10) Pub. No.: US 2024/0272138 A1
(43) Pub. Date: Aug. 15, 2024

(54) NUCLEIC ACID NANOPORE WITH ENHANCED SENSING FUNCTIONALITY

(71) Applicant: UCL BUSINESS LTD, London (GB)

(72) Inventors: Stefan HOWORKA, London (GB); Yongzheng XING, London (GB); Adam DOREY, London (GB)

(21) Appl. No.: 18/570,947

(22) PCT Filed: Jun. 17, 2022
(86) PCT No.: PCT/EP2022/066626

§ 371 Date: Mar. 18, 2024

(30) Foreign Application Priority Data
Jun. 18, 2021 (GB) 2108820.8
Apr. 8, 2022 (GB) 2205163.5

(51) Int. Cl. G01N 33/487; C12Q 1/6804; G01N 33/566

(52) U.S. Cl. CPC G01N 33/48721; C12Q 1/6804; G01N 33/566

(57) ABSTRACT

A sensing nucleic acid nanopore is provided. The nanopore possesses a geometry and wherein the nanopore defines a central lumen passing therethrough. The geometry of the nanopore is configured to accommodate all or a part of an analyte molecule within, or proximate to, the central lumen so as to optimize obstruction of the central lumen by the analyte molecule. Methods for enhancing binding of an analyte molecule to a membrane-spanning nanopore are provided. Membranes, sensor devices and methods for molecular sensing comprising the sensing nucleic acid nanopores are also provided.

### FIELD OF THE INVENTION

The present invention relates to novel membrane nanostructures and their uses. In particular it relates to wide-channel membrane nucleic acid nanopores in the applications of protein sensing and molecular gate creation.

### BACKGROUND OF THE INVENTION

The sudden and dramatic shut down of many aspects of normal society caused by the severe acute respiratory syndrome coronavirus 2 (SARS-CoV-2) pandemic in 2020 was in part due to the inability of many countries to roll out rapid and efficient track and trace arrangements. The swift levels of community transmission of this infection brought into sharp focus the over reliance of many public health systems upon ponderous laboratory-based diagnosis of infection with the preferred gold standard diagnostic method being the real-time reverse transcription-PCR (RT-PCR) test. While laboratory-based testing remains the ultimate benchmark for accuracy, limited levels of capacity and slow rates of scale up means that public health authorities cannot continue to rely on this approach when planning for future rapid response in times of acute need. The options for point-of-care diagnostics are largely limited to lateral flow serological assays that offer potential for rapid results via home reading or postal testing, however, concerns exist around test performance characteristics, particularly in early stage infection prior to onset of symptoms, as well as their poor positive predictive value when applied to a general population. There is a need, therefore, to provide alternative testing platforms that can provide high levels of accuracy at the point-of-care but which can be deployed and used quickly.

Nanopores are membrane spanning polymers and complexes that can define a perforation and thereby form a channel in a membrane that forms a partition between two fluids, typically liquids, suitably aqueous solutions, through which ions and certain molecules may pass. Membrane spanning nanopores composed of nucleic acid duplexes, in particular DNA duplexes, represent a possibility for developing sensing nanopores.

DNA nanopores have recently been obtained from a structural core of six hexagonally arranged, interlinked DNA duplexes that enclose a hollow channel (see, for example, Douglas S. M., Marblestone A. H., Teerapittayanon S., Vazquez A., Church G. M., Shih W. M. Nucleic Acids Res. 37, 5001-5006 (2009); Zheng J., et al. Nature 461, 74-77 (2009); Rothemund P. W. Nature 440, 297-302 (2006); Fu J., et al. Nat. Nanotechnol. 9, 531-536 (2014); Burns J. R., et al. Angew. Chem. Int. Ed. 52, 12069-12072 (2013); and Seifert A., Göpfrich K., Burns J. R., Fertig N., Keyser U. F., Howorka S. ACS Nano 9, 1117-1126 (2015)). Membrane insertion was achieved through equipping the pores exterior with hydrophobic lipid anchors. The modular construction principle for DNA nanopores has enabled customized pore diameter (Göpfrich et al, Nano. Lett., 15(5), 3134-3138 (2015); WO 2013/083983) and installation of a controllable gate to regulate transport (Burns J. R., Seifert A., Fertig N., Howorka S. A., Nat. Nanotechnol. 11, 152-156 (2016)). Circular nanotubes synthesized from DNA have also been described (Zheng et al. J. Am. Chem. Soc., 136, 10194-10197 (2014)).

To be useful as a sensor for large biomolecular analytes such as circulating antibodies, cancer or pathogen markers, suitable membrane channels formed by a nucleic acid nanopore should meet certain criteria, namely:
1) the channel lumen should be at least about 3 nm wide to accommodate the large biomolecule molecules inside or close to the opening of the channel lumen; binding of the large biomolecule close to or within the channel results in higher read-out sensitivity than when analytes bind in the general vicinity of the pore entrance;
2) the pores should be structurally defined and resistant to flex or distortion to attain a constant base level in the electrical read-out (i.e. reduce background noise); and
3) the pore dimensions should be easily tuneable to adapt them to different biomolecule sizes.

Membrane spanning nucleic acid nanopores with a minimum internal pore width of several nanometers are described in WO-2018/011603-A1, and also in WO2020/025974-A1.

Nevertheless, the diversity of size and form of potential biomolecular analytes makes the design and implementation of accurate point-of-care sensor technologies challenging. As has been evident from the response to the SARS-CoV-2 pandemic, global public health authorities need to reassure an anxious population that the testing technologies employed are both reliable and accurate. The risk associated with high levels of false negative or false positive test results are the main barrier to adoption of rapid point-of-care diagnostics. Yet the promise, for example, of being able to stop a future localized epidemic in its tracks before it becomes a pandemic of global proportions is largely dependent on the track and trace testing capacity at the focal point of the infection. Likewise, many major public health programs rely upon local testing capacity, which can be problematic in developed and developing nations alike. Hence, there exists need to further improve the accuracy and reliability of rapid point-of-care diagnostic technologies to meet these profound needs.

These and other uses, features and advantages of the invention should be apparent to those skilled in the art from the teachings provided herein.

### SUMMARY OF THE INVENTION

The present invention relates to the design and implementation of novel nucleic acid nanopore geometry to match that of one or more analytes that are being tested for. This differs from simple tethering of a binding molecule to a generic round or polygonal pore and relies on a synergistic interaction of binding moiety with the geometry of the pore that cooperates to improve the quality of information derivable from an electrical signal passing through the nanopore at the time the analyte binds.

A first aspect of the invention provides a sensing nucleic acid nanopore, wherein the nanopore possesses a geometry and wherein the nanopore defines a central lumen passing therethrough and wherein the geometry of the nanopore is configured to accommodate all or a part of an analyte molecule within or proximate to the central lumen so as to optimize obstruction of the central lumen by the analyte molecule.

A second aspect of the invention provides for a sensing nucleic acid nanopore comprising:
a nucleic acid nanopore, wherein the nanopore defines a central lumen passing therethrough, and wherein the central lumen has a cis-facing first open end that defines a first aperture and a trans-facing second open end that defines a second aperture; and
an analyte binding moiety, wherein the analyte binding moiety comprises an affinity binding molecule, and wherein the analyte binding moiety is located within the central lumen between the first and second apertures;
   wherein the nanopore has a tertiary structure and the tertiary structure of the nanopore is configured to conform to a three-dimensional shape of at least a portion of an analyte molecule.

A third aspect of the invention provides for a membrane comprising at least one nanopore as described herein.

A fourth aspect of the invention provides for a sensor device comprising a sensing nucleic acid nanopore as described herein.

A fifth aspect of the invention provides a method for enhancing binding of an analyte molecule to a membrane-spanning nucleic acid nanopore, the method comprising:
Selecting a geometry of the nanopore so that it conforms to the three dimensional shape of at least a portion of the analyte molecule so that the nanopore is able to accommodate the analyte molecule and thereby to modify a measurable electrical or optical signal through or across the nanopore; and
   manufacturing a nucleic acid nanopore to have the required geometry.

A sixth aspect of the invention provides for a nucleic acid nanopore obtained via the method of the fifth aspect of the invention.

A seventh aspect of the invention provides for a nucleic acid nanopore obtainable via the method of the fifth aspect of the invention.

An eighth aspect of the invention provides a method for molecular sensing, the method comprising:
providing the sensor device as described herein;
contacting the nanopore with an analyte molecule and establishing a flow of ions through the at least one nanopore or an electron flow across the nanopore; and
measuring an electrical signal across the nanopore,
   wherein the molecular sensing comprises analyte molecule detection or characterisation, wherein a change in the electrical measurement is indicative of the analyte molecule.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim although not originally claimed in that manner.

### BRIEF DESCRIPTION OF THE DRAWINGS

One or more embodiments of the invention will now be described, by way of example only, with reference to the accompanying drawings, in which:

FIG. 1 is a representation of a nucleic acid nanopore of a type used in an embodiment of the invention (A) shows a planar view of the nanopore with orientation of the main scaffold strands shown as cylinders; (B) shows a reverse view with hydrophobic anchor placement visible—in this chase cholesterol shown as cones; (C) the nanopore structure is in the process of insertion into a lipid membrane.

FIG. 2 is a representation of an embodiment of the invention (A) depicts a virion particle, such as SARS-CoV-2, the particle displays a surface protein, such as a spike protein, (i) that is recognised by an immunoglobulin molecule (ii); (B) shows a NTA-His6 linker suitable for attaching all or a part of the spike protein (i) to the nucleic acid nanopore; (C) depicts a plan view of nucleic acid nanopore that is of triangular configuration with the spike protein tethered via the linker so as to become a binding moiety that is orientated inwardly into the central lumen of the nanopore, the antibody (ii) is able to fit within the lumen due to the complementary geometry of the pore and to interact with the binding moiety.

FIG. 3 shows a side sectional representation of a nanopore embedded within a membrane or solid state partition according to an embodiment of the invention, (A) a binding moiety is located within a central lumen of the nanopore, an open end of the nanopore—the aperture—is depicted as region x; (B) a binding moiety is located proximate to the aperture extending outwardly above the lumen.

FIG. 4 shows a representation of an embodiment of the invention in plan view of a triangular nanopore located within a membrane or solid state partition, a binding moiety shown as a star (★) is located within the lumen; (B) an analyte, shown as an IgG type immunoglobulin is accommodated within the geometry of the lumen and binds to the binding moiety.

FIG. 5 shows a representation of an embodiment of the invention in plan view of a regular polygonal nanopore located within a membrane or solid state partition, a binding moiety shown as a star (★) is located within the lumen; (B) an analyte, shown as multidomain polypeptide complex is accommodated within the geometry of the lumen and binds to the binding moiety.

FIG. 6 shows analysis of a triangular membrane-embedded DNA nanopore with 20 nm side length (Tri-20): (a) Example figure showing raw current trace at 10 mV (b) current-voltage (IV) curves displaying the averages and standard errors from 5 independent single-channel current traces, (c) conductance histogram obtained from 5 independent insertions at 20 mV. The recordings were acquired in 1 M KCl, 10 mM HEPES, PH 7.4.

FIG. 7 shows analysis of 20 nm triangular membrane embedded DNA nanopore carrying a SARS CoV 2 spike protein via a metal chelate bridge according to one embodiment of the present invention (Tri-20-Spike pore): (a) Example figure showing raw current trace at 10 mV, (b) current-voltage (IV) curves displaying the averages and standard errors from 3 independent single-channel current traces, (c) conductance histogram obtained from 3 independent insertions at 20 mV. The recordings were acquired in 1 M KCl, 10 mM HEPES, pH 7.4.

FIG. 8 shows analysis of the nanopore of FIG. 7 upon addition of anti-spike antibody (a) example trace showing an open channel before addition of 44.5 nM SARS-CoV-2 anti-spike antibody, the arrow indicates addition of antibody—upon addition blockades occur, (b) blockades have been analysed with regards to their dwell times and average block.

FIG. 9 shows the Tri-20-Spike pore inserted into schematically drawn membrane arrays of a high-throughput MinION sensor device. The MinION is 10 cm in length.

FIG. 10 shows single-channel current traces of Tri-20-Spike after insertion into MinION membranes within the MinION device, at −50 mV before and after (after gap) addition of 44.5 nM SARS-CoV-2 anti-spike antibody.

FIG. 11 shows a scatter plot of antibody binding events of Tri-20-Spike with 44.5 nM SARS-CoV-2 anti-spike antibody, represented in relation to the T_{off} and relative amplitude, A/I_{O}, at −40 mV.

FIG. 12 shows analysis of the Tri-20 and Tri-20-spike nanopores using the MinION device. Analysis via single-channel current recordings of (a) Tri-20 and (b) Tri-20-Spike in high (red) and low (blue) conductance states, in 1 M salt conditions, (i) representative single-current traces recorded at −30 mV, (ii) current-voltage (IV) graphs for voltages ranging from −60 mV to +60 mV in 20 mV steps, and (iv) conductance histograms obtained at −20 mV.

FIG. 13 shows analysis of SARS-CoV-2 antibody binding to the Tri-20-Spike nanopore using the MinION device from Oxford Nanopore Technologies. Example traces showing pore before antibody addition (i) and the same pore after the addition of 44.5 nM antibody (ii) at (a), −100 mV, (b), −80 mV, (c), −70 mV, (d), −50 mV, (e), −40 mV, (f), −30 mV, (g), −20 mV. The traces are compiled from 3 independent pore recordings.

FIG. 14 shows single-channel current traces of DNA nanopore Tri-20-link, a version of the Tri-20 pore with the linker region but no SARS-CoV-2 spike protein, obtained using the Orbit Mini device after addition of (a) 0 nM, (b) 47 nM (c) 88 nM and (d) 218 nM SARS-CoV-2 antibody. Traces were recorded in 1M KCl, 10 mM HEPES PH 7.4 at +20 mV.

FIG. 15 shows single-channel current traces of DNA nanopore Tri-20-Spike obtained using the Orbit Mini device after addition of (a) 0 nM, (b) 47 nM (c) 88 nM and (d) 218 nM non-specific (anti-biotin) antibody. Traces were recorded in 1M KCl, 10 mM HEPES pH 7.4 at +20 mV.

### DETAILED DESCRIPTION OF THE INVENTION

Unless otherwise indicated, the practice of the present invention employs conventional techniques of chemistry, molecular biology, microbiology, recombinant DNA technology, and chemical methods, which are within the capabilities of a person of ordinary skill in the art. Such techniques are also explained in the literature, for example, M. R. Green, J. Sambrook, 2012, Molecular Cloning: A Laboratory Manual, Fourth Edition, Books 1-3, Cold Spring Harbor Laboratory Press, Cold Spring Harbor, NY; Ausubel, F. M. et al. (Current Protocols in Molecular Biology, John Wiley & Sons, Online ISSN:1934-3647); B. Roe, J. Crabtree, and A. Kahn, 1996, DNA Isolation and Sequencing: Essential Techniques, John Wiley & Sons; J. M. Polak and James O′D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press; Synthetic Biology, Part A, Methods in Enzymology, Edited by Chris Voigt, Volume 497, pages 2-662 (2011); Synthetic Biology, Part B, Computer Aided Design and DNA Assembly, Methods in Enzymology, Edited by Christopher Voigt, Volume 498, Pages 2-500 (2011); RNA Interference, Methods in Enzymology, David R. Engelke, and John J. Rossi, Volume 392, Pages 1-454 (2005). Each of these general texts is herein incorporated by reference.

Prior to setting forth the invention, a number of definitions are provided that will assist in the understanding of the invention.

As used herein, the term ‘comprising’ means any of the recited elements are necessarily included and other elements may optionally be included as well. ‘Consisting essentially of’ means any recited elements are necessarily included, elements that would materially affect the basic and novel characteristics of the listed elements are excluded, and other elements may optionally be included. ‘Consisting of’ means that all elements other than those listed are excluded. Embodiments defined by each of these terms are within the scope of this invention.

The term ‘membrane’ as used herein is an enclosing or separating selectively-permeable boundary, partition, barrier or film. The membrane has two sides or surfaces which may be named the cis and trans side respectively. The membrane is thin (i.e. has a thickness substantially less than its width and length) allowing it to be spanned by the nanopore. In the context of the present invention, the membrane thickness is typically in the nanometre (10-9 metre) range. The arrangement of the membrane is not limited and may assume any form, for example, a liposome, and a vesicle or as a planar or a non-planar sheet. Specific examples of membranes useful in the present invention include lipid bilayers, polymeric films, or solid state substrates.

The term ‘solid state membrane’ or ‘solid state substrate’ as used herein refers to a membrane or partition formed from a solid state substance—i.e. not a semi-fluid membrane—in which one or more holes/bores are provided. One or more nanopores may be positioned within the respective one or more holes/bores disclosed for example in U.S. Pat. No. 8,828,211, hereby incorporated by reference. The solid state membrane may comprise either or both of organic and inorganic materials, including, but not limited to, microelectronic materials, whether electrically conducting, electrically semiconducting, or electrically insulating, including materials such as II-IV and III-V materials, oxides and nitrides, such as silicon nitride, Al₂O₃, and SiO₂, Si, MoS₂, solid state organic and inorganic polymers such as polyamide, plastics such as Teflon®, or elastomers such as two-component addition-cure silicone rubber, and glasses. A membrane may be formed from monatomic layers, such as graphene, or layers that are only a few atoms thick such as those disclosed in U.S. Pat. No. 8,698,481, and U.S. Patent Application Publication 2014/174927, both hereby incorporated by reference. More than one layer of material can be included, such as more than one graphene layer, as disclosed in US Patent Application Publication 2013/309776, incorporated herein by reference. Suitable silicon nitride membranes are disclosed in U.S. Pat. No. 6,627,067, and the membrane may be chemically functionalized, such as disclosed in U.S. Patent Application Publication 2011/053284, both hereby incorporated by reference. Such a structure is disclosed for example in U.S. Pat. No. 8,828,211, hereby incorporated by reference. The internal walls of the holes/bores may be coated with a functionalised coating, such as disclosed in published application WO 2009/020682. The one or more holes/bores may be hydrophobic or provided with a hydrophobic coating to assist the provision of the one or more nanopores in the respective one or more holes/bores. Suitable methods for providing holes/bores in solid state substrates are disclosed in published applications WO 03003446 and WO 2016/187519.

The term ‘modular’ as used herein refers to the use of one or more units, or modules, to design or construct a whole or part of a larger system. In the context of the present invention it refers to the use of individual modules, sub-units or building blocks to construct a nanopore. The modules may be each the same or the modules may be different. To form the nanopore, the individual modules may be connected or inter-linked to one or more other modules. The means of connection between modules may be by chemical or physical means, such as covalent or non-covalent chemical bonding or by electrostatic or other attractive forces. Alternatively, or in addition, the means of connection may be via an additional module, bracing member, portion or linkage. The modular design of a nanopore may comprise a frame or framework of modules, and additional, typically smaller, sub-modules that connect, or support the frame, acting as struts or bracing members. The modules may span the membrane to enable formation of the channel of the nanopore; the sub-modules do not generally span the membrane and are intended only as structural support in the nanopore. Some modules may sit on a surface of the membrane in order to stabilise membrane insertion of membrane spanning modules. Such surface located modules may adopt a raft-like configuration and serve as location points for one or more anchors. The design of the modules and sub-modules, or how they connect, may be chosen to support and strengthen the formed channel of the nanopore such that it maintains its shape and conformational integrity when inserted in a membrane. The modules or sub-modules may be formed of nucleic acids, typically DNA. Each individual unit may be assembled by DNA origami techniques described elsewhere herein using suitably selected scaffold and staple strands.

The term ‘nucleic acid’ as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may include DNA and RNA, and are typically manufactured synthetically, but may also be isolated from natural sources. Nucleic acids may further include modified DNA or RNA, for example DNA or RNA that has been methylated or that has been subject to chemical modification, for example 5′-capping with 7-methylguanosine, 3′-processing such as cleavage and polyadenylation, and splicing, or labelling with fluorophores or other compounds. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Hence, where the terms ‘DNA’ and ‘RNA’ are used herein it should be understood that these terms are not limited to only include naturally occurring nucleotides. Sizes of nucleic acids, also referred to herein as ‘polynucleotides’ are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 100 nucleotides in length are typically called ‘oligonucleotides’.

As used herein, the terms ‘3″ (‘3 prime’) and ‘5″ (‘5 prime’) take their usual meanings in the art, i.e. to distinguish the ends of polynucleotides. A polynucleotide has a 5′ and a 3′ end and polynucleotide sequences are conventionally written in a 5′ to 3′ direction. The term ‘complements of a polynucleotide molecule’ denotes a polynucleotide molecule having a complementary base sequence and reverse orientation as compared to a reference sequence.

The term ‘duplex’ is used herein refers to double-stranded DNA, meaning that the nucleotides of two complimentary DNA sequences have bonded together and then coiled to form a double helix.

According to the present invention, homology to the nucleic acid sequences described herein is not limited simply to 100%, 99%, 98%, 97%, 95% or even 90% sequence identity. Many nucleic acid sequences can demonstrate biochemical equivalence to each other despite having apparently low sequence identity. In the present invention homologous nucleic acid sequences are considered to be those that will hybridise to each other under conditions of low stringency (Sambrook J. et al, Molecular Cloning: a Laboratory Manual, Cold Spring Harbor Press, Cold Spring Harbor, NY).

As used herein, the term ‘nanostructure’ refers to a predesigned two or three dimensional molecular structure typically comprised from a biopolymer, suitably a naturally or non-naturally occurring nucleic acid, which structure has at least one dimension or an aspect of its geometry that is within the nanoscale (i.e. 10⁻⁹ metres). Nanoscale structures suitably have dimensions or geometry of less than around 100 nm, typically less than 50 nm, and most suitably less than 20 nm. Nanoscale structures suitably possess dimensions or geometry greater than around 0.1 nm, typically greater than around 1 nm, and optionally greater than around 2 nm. Assembly of nucleic acid nanostructures may occur spontaneously in solution, such as by heating and cooling a mixture of DNA strands of preselected sequences, or may require presence of additional co-factors including, but not limited to, nucleic acid scaffolds, nucleic acid aptamers, nucleic acid staples, co-enzymes, and molecular chaperones. Where desired nanostructures result from one or more predesigned spontaneously self-folding nucleic acid molecules, such as DNA, this is typically referred to as nucleic acid ‘origami’. Rational design and folding of DNA to create two dimensional or three dimensional nanoscale structures and shapes is known in the art (e.g. Rothemund (2006) Nature 440, 297-302). In the classical scaffold-and-staple approach, one or more long biogenic scaffold strand component(s) is folded into a defined DNA nanostructure with a staple component consisting of shorter synthetic staple oligonucleotides. Suitably scaffold structures may be based off M13 or phix174 sequences, which a plurality of smaller staple and linker sequences configured to achieve the desired three-dimensional nanostructural geometry.

Classical DNA nanostructures are formed of bundles of parallel aligned DNA duplexes that are arranged into polygons that enclose a channel and puncture a membrane bilayer. However, a challenge with nucleic acid nanostructures remains that the strong net negative charge of the phosphodiester background hinders insertion into amphipathic and hydrophobic planar membranes. As a result, this has often favoured their use in solid state contexts, as nanofunnels attached to or sited within a nanoscale hole or bore in a substrate. A problem associated with such arrangements, however, is that they can often exhibit high levels of ionic leakage in sensor applications due to poor fit between the DNA duplex and the nanoscale hole. Ionic leakage is much reduced when nanopores are embedded within semi-fluid membranes which surround the pore.

The nucleic acid sequences that form the nanostructures will typically be manufactured synthetically, although they may also be obtained by conventional recombinant nucleic acid techniques. DNA constructs comprising the required sequences may be comprised within vectors grown within a microbial host organism (such as E. coli). This would allow for large quantities of the DNA to be prepared within a bioreactor and then harvested using conventional techniques. The vectors may be isolated, purified to remove extraneous material, with the desired DNA sequences excised by restriction endonucleases and isolated, such as by using chromatographic or electrophoretic separation.

The term ‘amino acid’ in the context of the present invention is used in its broadest sense and is meant to include naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term ‘amino acid’ further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as ‘functional equivalents’ of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

A ‘polypeptide’ is a polymer of amino acid residues joined by peptide bonds, whether produced naturally or in vitro by synthetic means. Polypeptides of less than around 12 amino acid residues in length are typically referred to as ‘peptides’ and those between about 12 and about amino acid residues in length may be referred to as ‘oligopeptides’. The term ‘polypeptide’ as used herein denotes the product of a naturally occurring polypeptide, precursor form or proprotein. Polypeptides can also undergo maturation or post-translational modification processes that may include, but are not limited to: glycosylation, proteolytic cleavage, lipidization, signal peptide cleavage, propeptide cleavage, phosphorylation, and such like. The term ‘protein’ is used herein to refer to a macromolecule comprising one or more polypeptide chains.

The term ‘folded protein’ as used herein refers to a protein that has acquired some three-dimensional shape after translation of the polypeptide chain from which it is formed (the primary structure). The term may refer to the secondary structure of the protein which is typically the first stage of the folding process where local three-dimensional structures are formed, for example, alpha helices or beta sheets. The term may more typically refer to the tertiary structure of a protein where the secondary structures of the protein have folded to stabilise the structure through hydrophobic or covalent interactions. The term also encompasses proteins having a quaternary structure where one or more protein subunits are assembled. As appropriate, the folded protein may also be termed the ‘native’ protein structure, and may be the form of the protein that exhibits its biological function.

The term ‘interior width’ when used herein refers to a straight distance spanning the interior of the channel (e.g. the lumen) from a location on an interior face of one wall to an interior face of an opposing wall in a plane perpendicular to the longitudinal axis of the channel (i.e. in cross section). The interior width of the channel may be constant along its longitudinal axis or it may vary due to the presence of one or more constrictions. The ‘minimum interior width’ is a minimum interior width along the longitudinal axis of the channel between an entrance and an exit of the channel. The minimum interior width of a channel defines the maximum size of an object, such as an analyte, that may pass through the channel. In instances where the lumen is circular or is comprised of a regular polygonal shape in cross section, the interior width may correspond to the internal diameter of the lumen. However, it will be appreciated that in embodiments of the invention the configuration of the nanopore will be such that the nanopore may not have a lumen with a regular polygonal or circular shape in cross section such that there may be several interior widths.

As used herein the term ‘hydrophobic’ refers to a molecule having apolar character including organic molecules and polymers. Examples are saturated or unsaturated hydrocarbons. The molecule may have amphipathic properties.

As used herein, the term ‘hydrophobically-modified’ relates to the modification (joining, bonding or otherwise linking) of a polynucleotide strand with one or more hydrophobic moieties. A ‘hydrophobic moiety’ as defined herein is a hydrophobic organic molecule. The hydrophobic moiety may be any moiety comprising non-polar or low polarity aliphatic, aliphatic-aromatic or aromatic chains. Suitably, the hydrophobic moieties utilised in the present invention encompass molecules such as long chain carbocyclic molecules, polymers, block co-polymers, and lipids. The term ‘lipids’ as defined herein relates to fatty acids and their derivatives (including tri-, di-, monoglycerides, and phospholipids), as well as sterol-containing metabolites such as cholesterol. The hydrophobic moieties comprised within the embodiments of the present invention are capable of forming non-covalent attractive interactions with phospholipid bilayers, such as the lipid-based membranes of cells and act as membrane anchors for the nanopore. According to certain embodiments of the present invention suitable hydrophobic moieties, such as lipid molecules, possessing membrane anchoring properties may include sterols (including cholesterol, derivatives of cholesterol, phytosterol, ergosterol and bile acid), alkylated phenols (including methylated phenols, dolichols and tocopherols), flavones (including flavanone containing compounds such as 6-hydroxyflavone), saturated and unsaturated fatty acids (including derivatives such as lauric, oleic, linoleic and palmitic acids), and synthetic lipid molecules (including dodecyl-beta-D-glucoside). The anchors for the polymer membrane may be the same as for lipid bilayers or they may be different. The specific hydrophobic moiety anchor may be selected based on the binding performance of the membrane chosen.

The nanopore of the present invention may comprise at least one, or optionally two or more membrane anchors that act to attach or connect or anchor the hydrophilic DNA nanopore to the generally hydrophobic membrane (lipid bilayer or polymer). The anchors may be hydrophobic anchors, and may be further selected from the group consisting of: a lipid anchor; and a porphyrin. The lipid anchors are attached to the pore, or comprised within modules that form part of overall the nanostructure of the pore. Modules may be embedded within the membrane (i.e. membrane spanning) or may be located on a cis or trans surface of the membrane and associated with an aperture of the membrane spanning nanopore. Suitably attachment is via DNA oligonucleotides or DNA polynucleotides that carry the at least one lipid anchor, suitably cholesterol, at the 5′ or 3′ terminus. Polynucleotides or oligonucleotides may be functionalized using a modified phosphoramidite in the strand synthesis reaction, which is easily compatible for the addition of reactive groups, such as cholesterol and lipids, or attachment groups including thiol and biotin. Enzymic modification using a terminal transferase can also be used to incorporate an oligonucleotide, which incorporates a modification such as an anchor, to the 3′ of a single stranded nucleic acid (e.g. ssDNA). These lipid-modified anchor strands may hybridize via ‘adaptor’ oligonucleotides to corresponding sections of the DNA sequence forming the scaffold section of the pore. Alternatively, the lipid anchors are assembled with the pore using lipid-modified oligonucleotides that contribute as either the scaffold or staple strands. A combination of approaches to anchoring using at least one, or optionally two or more membrane anchors may also be adopted wherein anchors are incorporated into one or all of a scaffold strand, a staple strand and an adaptor oligonucleotide. Cholesterol has been found to be a particularly suitable lipid for use as an anchor in the present invention. The use of other lipids as anchors is contemplated, although it may be expected that there is a particular preference for a particular lipid, and a given number of membrane anchors, for a given membrane.

In an alternative embodiment of the invention, the hydrophobic modification is comprised within one or more synthetic nucleic acids (XNAs) incorporated into the nanopore structure itself. According to a specific embodiment of the invention, the nanopore comprises at least one hydrophobic anchor that is comprised of a polynucleotide strand and at least one hydrophobic anchor molecule, wherein the at least one hydrophobic anchor molecules are:
a. attached to and spaced substantially equidistantly about the periphery of the nanopore, and wherein the at least one hydrophobic anchor molecules are orientated radially outwardly from the longitudinal axis of the central lumen of the nanopore; and/or
b. attached to a membrane-facing side of the nanopore or a portion thereof such that the at least one hydrophobic anchor molecules are orientated to interact with and/or extend perpendicularly into the membrane once inserted.

In an optional embodiment of the invention, the nanopore comprises at least four hydrophobic anchor molecules.

The membrane in which the nanopore of the present invention may be inserted may be of any suitable type. Depending on the intended use, the membrane may be a lipid bilayer or a polymer sheet or film. The membrane is suitably an amphiphilic layer. The amphiphilic layer may be a monolayer or a bilayer. An amphiphilic layer is a layer formed from amphiphilic molecules which have both hydrophilic and lipophilic properties. The amphiphilic molecules may be synthetic or naturally occurring. The lipophilic properties of the molecules comprising the membrane promote anchoring by lipid anchors or other hydrophobic anchoring regions of the nanopore. It is surprising that nucleic acid nanostructures of the invention are able to be inserted successfully into membranes at all given that the hydrophobic nature of the membrane leads to repulsion of the predominantly negatively charged DNA backbone. It might be expected that the nanostructures of the invention would simply form clustered aggregates on one surface of the membrane as is often the case with complex nucleic acid nanostructures that fail to insert successfully into an amphiphilic mono- or bilayer. The ability of the nanostructures to be embedded within such membranes is, therefore, surprising and most unexpected.

In a specific embodiment of the invention the amphiphilic layer may be a lipid bilayer. The lipid composition may comprise naturally-occurring lipids such as phospholipids and bipolar tetraether lipids, and/or artificial lipids. The lipids typically comprise a head group, an interfacial moiety and two hydrophobic tail groups which may be the same or different. Suitable head groups include, but are not limited to, neutral head groups, zwitterionic head groups, negatively charged head groups and positively charged headgroups. The head group or the tail group of the lipids may be chemically-modified.

Proprietary and non-proprietary synthetic polymer films or sheets are widely used in ‘chip-based’ nanopore sequencing and analytical sensor applications such as the MinION® system sold by Oxford Nanopore Technologies®; the GS FLX+® and the GS Junior® System sold by Roche®; the HiSeq®, Genome Analyzer IIx®, MiSeq® and the HiScanSQ® systems sold by Illumina®; the Ion PGM® System and the Ion Proton System® sold by Life Technologies; the CEQ® system sold by Beckman Coulter®; and the PacBio RS® and the SMRT® system sold by Pacific Biosciences®. The ability of nanopores to insert successfully into polymer membranes of this type allows these systems to be adapted for diverse folded protein sensing applications, for example.

Non-naturally occurring amphiphiles and amphiphiles which form an amphiphilic membrane layer are known in the art and include, for example, block copolymers (Gonzalez-Perez et al., Langmuir, 2009, 25, 10447-10450). The block copolymer may be a diblock (consisting of two monomer sub-units), but may also be constructed from more than two monomer sub-units to form more complex arrangements that behave as amphiphiles. The copolymer may be a triblock, tetrablock or pentablock copolymer. The membrane may be chosen one of the membranes disclosed in PCT/GB2013/052767, hereby incorporated by reference in its entirety. The amphiphilic molecules may be chemically-modified or functionalised to facilitate coupling an insertion of the nanostructure. The membrane can comprise both a lipid and an amphiphilic polymer such as disclosed in PCT/US2016/040665.

Polymer-based membrane may be formed of any suitable material. Typically, synthetic membranes are composed of amphiphilic synthetic block copolymers. Examples of hydrophilic block copolymers are poly(ethylene glycol) (PEG/PEO) or poly(2-methyloxazoline), while examples of hydrophobic blocks are polydimethylsiloxane (PDMS), poly(caprolactone (PCL), poly(lactide) (PLA), or poly(methyl methacrylate) (PMMA). In embodiments, the polymer membrane used may be formed from the amphiphilic block copolymer poly 2-(methacryloyloxy)ethyl phosphorylcholine-b-disisopropylamino) ethyl methacrylate (PMPC-b-PDPA). DNA nanopores may be inserted into the walls of such polymersomes through incubation. Without wishing to be bound by theory, it is believed that one process of insertion broadly involves first steps of membrane tethering, followed by second steps of orientation of the DNA pore relative to the membrane to achieve complete insertion. This however requires lipid membrane anchors to be comprised within, or at least attached to, the pores, without which insertion does not take place. For alternative configurations of the invention, particularly where nucleic acid analogues (e.g. XNAs) comprise some or all of the backbone of the scaffold and/or staple strands, it is envisaged that more complex insertional dynamics may be observed. It will be appreciated that where the scaffold and staple strands incorporate synthetic biopolymer backbone constituents capable of mediating a hydrophobic interaction with a membrane, the insertion process may or may not involve initial stages of coplanar alignment with the membrane followed by a phase of insertion.

The membrane is typically planar, although in certain embodiments it may be curved or shaped. Amphiphilic membrane layers may also be supported. Suitably the membrane is a lipid bilayer or monolayer. Methods for forming lipid bilayers are known in the art such as disclosed in International Application Number PCT/GB2008/000563. Lipid bilayers are commonly formed by the method of Montal and Mueller (Proc. Natl. Acad. Sci. USA., 1972; 69: 3561-3566).

In another embodiment of the invention, the membrane may comprise a solid state layer. Solid state layers can be formed from organic and/or inorganic materials including, but not limited to, microelectronic materials, insulating materials such as Si₃N₄, Al₂O₃, and SiO, glasses, organic and inorganic polymers such as polyamide and plastics. The solid state layer may be formed from graphene such as disclosed in PCT/US2008/010637. The membrane may be provided with one or more through-holes of nanometre scale dimensions extending from one side of the membrane to the other. The internal walls of the solid state hole may be coated with a lipid such as disclosed in US2017/0023544 or chemically functionalized such as disclosed in PCT/US2008/063066, so as to facilitate suitable anchoring of the nanostructures of the invention to the solid state layer.

In a specific embodiment of the present invention, the membrane may be selected from the group consisting of: a membrane comprising a semi-fluid membrane formed of polymers; and a solid state membrane. In an optional embodiment of the present invention, the polymer forming the semi-fluid membrane may be composed of amphiphilic synthetic block copolymers, suitably selected from hydrophilic copolymer blocks and hydrophobic copolymer blocks. In a further embodiment of the present invention, the membrane is in the form of a vesicle, a micelle, a planar membrane or a droplet. In an alternative embodiment of the present invention, the solid state membrane may be formed of a material selected from the group consisting of: Group II-IV and III-V oxides and nitrides, solid state organic and inorganic polymers, plastics, elastomers, and glasses.

The nanopore according to an embodiment of the present invention is a membrane or solid state substrate spanning nucleic acid nanostructure that is embedded within and, at least a portion thereof, is oriented substantially coplanar to a membrane or partition surface. In an embodiment of the invention, the nanopore is located in a membrane or substrate in a manner akin to a grommet or eyelet mounted in a planar or curved sheet material. Hence, in a specific embodiment of the invention the nucleic acid nanostructure of the invention is defined as a nanoscale grommet or eyelet, this is irrespective of the shape of the channel(s) which may be circular or polygonal. According to this embodiment of the invention a majority of the nanostructure of the nanopore is embedded within the membrane compared to the proportion of the nanostructure extending outside of the membrane. In a further embodiment of the invention the nanostructure comprises one or more modules that may extend radially from one or both sides of the pore but which are substantially co-planar with and sit upon a surface of the membrane (e.g. a cis or trans surface—or both). In such an embodiment a module may form a surface associated raft structure that radiates outwardly from an aperture in the pore.

Suitably the nanopores of the invention comprise one or more polynucleotide strands that provide a functional scaffold component, wherein the polynucleotide strands comprised within the scaffold component include a polynucleotide backbone; and a plurality of polynucleotide strands that provide a plurality of functional staple components. The scaffold strand(s) cooperate with and hybridise to the plurality of staple polynucleotide strands—e.g. via appropriate Watson-Crick base pairing hybridisation—in order to form a three-dimensional configuration of the nanopore. It is desirable that the nanopore assembles into an annular, elliptical, regular or irregular polygonal structure that is able to be embedded within a membrane or partition surface such that a majority of the 5′ to 3′ orientation of the staple component(s) is coplanar with the membrane. Hence within modules embedded within or coplanar with—e.g. lying upon—the membrane a majority of the 5′ to 3′ orientation of the staple component(s) of the modules are coplanar with the orientation of the membrane. This contrasts with nanostructures comprised of bundles of nucleic acids that are orientated 5′ to 3′ perpendicularly—e.g. in bundles—that pass through and extend outwardly from the membrane.

The nanopores of the present invention may be assembled via the ‘scaffold-and-staple’ approach. In this important route to nucleic acid nanostructures, DNA is utilized as a building material in order to make nanoscale three dimensional shapes. Assembly of these complex nanostructures from a plurality of un-hybridized linear molecules is typically referred to as ‘DNA origami’. The DNA origami process generally involves the folding of the one or more elongate, ‘scaffold’ DNA strands into a particular shape using a plurality of rationally designed ‘staple’ DNA strands. The scaffold strand can have any sufficiently non-repetitive sequence. The sequences of the staple strands are designed such that they hybridize to particular defined portions of the scaffold strands and, in doing so, these two components cooperate force the scaffold strands to assume a particular structural configuration. Methods useful in the making of DNA origami structures can be found, for example, in Rothemund, P. W., Nature 440:297-302 (2006); Douglas et al, Nature 459:414-418 (2009); Dietz et al, Science 325:725-730 (2009); and U.S. Pat. App. Pub. Nos. 2007/0117109, 2008/0287668, 2010/0069621 and 2010/0216978, each of which is incorporated by reference in its entirety. Staple sequence design can be facilitated using, for example, CaDNAno software, available at http://www.cadnano.org or the DAEDALUS online platform, available at http://daedalus-dna-origami.org.

In embodiments of the invention the staple and/or scaffold components further comprise a plurality of hydrophobic membrane anchor molecules that are attached thereto. The hydrophobic anchors (or portions of the sequence) facilitate insertion of the nanopore into a curved or planar membrane such that the orientation of a major portion of the first scaffold polynucleotide strand is substantially parallel to the surface of the membrane and wherein the first scaffold polynucleotide strand is embedded within and is substantially coplanar with the membrane. In referring to ‘a major portion’ of a scaffold polynucleotide stand it is meant that substantially more than 50%, suitably more than 60%, even greater than 70%, and as much as 90%, of the total length of that strand is orientated so that it is substantially coplanar with the membrane. In a specific embodiment the entire length of the scaffold strand, excepting crossovers and/or Holliday junctions necessary to impart three-dimensional structure on a resultant nanopore, is orientated so that it is substantially coplanar with the membrane. It will be appreciated that DNA origami techniques allow for variations of the embodied structures that, nevertheless, fall within the overall design constraints of the recited nanostructures of the present invention. By way of example, the scaffold strand may be comprised of a plurality of shorter scaffold strands that, when assembled following hybridisation to appropriate staple strands, will cooperate to serve in a manner equivalent to a unitary single length scaffold strand.

In embodiments, the nanopores of the present invention are formed or constructed from one or more modules. In embodiments, the nanopore may be formed of an arrangement of modules that forms a basic frame or framework. In embodiments, the modules of the frame are supported by additional, typically smaller, sub-modules that connect and support the structure of the frame. At least part of the module is intended to form at least part of the channel wall of the nanopore and therefore the module is designed and configured to span a membrane in which it is inserted. Sub-modules are intended for structural benefits only and therefore are not intended or configured to span the membrane. While each module of the frame, or the sub-modules, may be different, suitably, the modules of the frame are suitably substantially or completely identical units, as are the sub-modules forming the support, when present. In these embodiments, the modules and sub-modules are each formed of the same scaffold and staple DNA structure and assembled in the same way. The individual modules may be joined by DNA strands, the DNA strand either being integral with the module, or hybridised to each module. While any arrangement of the modules is contemplated, suitably, for circular or elliptical cross-section nanopores, the modules may be arranged to overlie each other to form a generally hollow stack or tower thereby forming a channel. Suitably, for nanopores having a polygonal cross-section, the modules are arranged such that they sit side by side in the plane of a membrane. A combination of the above arrangements is also contemplated. The modules may have tuneable side length (a side length in this context being defined as the longest dimension of the module parallel to the plane of the membrane), which when chosen with an appropriate final overall shape, allows for different sized and/or shaped nanopores, and different sized and/or shaped lumens within the channels of those pores to be prepared. The channels and the lumens defined thereby may be regularly or irregularly shaped. For example, the lumens defined by the channel may be a regular or irregular circle or polygon, such as a triangle, a quadrilateral (e.g. a square, a rectangle or a trapezoid), a pentagon, a hexagon, a heptagon, an octagon and so on. Alternatively, the channels may be an elongate circle (ellipse) or elongate polygon, such as rectangular, oblong or slot-shaped channels formed of 4 or more sides. Typically, the side length of the modules would be in the order of between 10 nm and 20 nm (FIG. 8). Suitably, the side length of the modules may be at least 2 nm, 3 nm, 4 nm, 5 nm, 6 nm, 7 nm, 8 nm, 9 nm or 10 nm, Suitably the side length of the modules may be at most 30 nm, 25 nm, 20 nm. The sizing of the sub-modules is determined by the spacing between the modules which is turn is determined by the shape of the pore and the size and number of modules employed. Suitably, the side length of the modules may be at least 0.5 nm, 1 nm, 1.5 nm, 2 nm, 2.5 nm, 3 nm, 3.5 nm, 4 nm or 5 nm, Suitably the side length of the modules may be at most 10 nm, 7.5 nm, or 5 nm.

As used herein, the term ‘substantially’ refers to the complete or nearly complete extent or degree of an action, characteristic, property, state, structure, item, or result. For example, an object that is ‘substantially’ coplanar with another object would mean that the object is either completely coplanar or nearly completely coplanar, perhaps varying by a few degrees of variation from complete conformity. The exact allowable degree of deviation from absolute completeness may in some cases depend on the specific context, as would be understood to the person of skill in the art. However, in general terms the nearness of conformity to the absolute will be such as to have the same overall result—e.g. functional equivalence—as if total conformity were achieved.

The three-dimensional configuration of the nanopores of the present invention defines at least one channel, suitably a single channel that spans the membrane, the channel having a lumen that has a minimum internal width of at least about 3 nm. Suitably, the nanopores of the present invention have a single channel located at least substantially centrally in the pore structure when viewed perpendicular to the plane of the membrane in which the pore is intended to reside. The channel defines the lumen that passes through the nanopore which is perpendicular to the planar axis defined by the membrane. The minimum opening, or aperture, of the channel in this cross-section (e.g. the minimum constriction) is suitable to facilitate a close fitting interaction with a folded protein or other analyte. Typically, the minimum opening is at least 3 nm, 6 nm, 6.5 nm, 7 nm, 7.5 nm, 8 nm, 8.5 nm, 9 nm, 9.5 nm, 10 nm, 11 nm, 12 nm, 13 nm, 14 nm, or 15 nm or more. Suitably the lumen is between around 10 nm and around 20 nm in width. Suitably, the maximum opening of the channel (i.e. minimum constriction) is at most 200 nm, 150 nm, 100 nm, 75 nm, 50 nm, 40 nm, 30 nm, 20 nm, 18 nm, 15 nm, 12 nm, or 10 nm. Suitably, the cross-sectional area of the minimum opening of the channel (i.e. minimum constriction) is at least 5 nm², 12 nm², 15 nm², 25 nm², 35 nm², 40 nm², 45 nm² or 50 nm² or more. Suitably, the cross-sectional area of the minimum opening of the channel (i.e. minimum constriction) is at most 35 000 nm², 25 000 nm², 15 000 nm², 10 000 nm², 5 000 nm², 1500 nm², 1000 nm², 750 nm², 500 nm², 250 nm², 100 nm², 50 nm², 30 nm², 20 nm² or 15 nm², 10 nm², 7 nm² or less.

In accordance with an embodiment of the present application, the nanopore defines a lumen that extends along a central axis of the nanostructure thereby defining at least a first and a second opening. The first and second openings may be referred to as apertures (e.g. first and second apertures) and permit fluid communication through the lumen of the pore. Suitably the first aperture is located on the cis side of the nanopore and the second aperture on the trans side. When the nanopore is embedded within a membrane or a solid-state substrate the fluid communication through the pore permits a measurable flow of electrically charged ions to pass through the pore—i.e. a measurable electrical current to pass across the membrane or substrate partition via the nanopore.

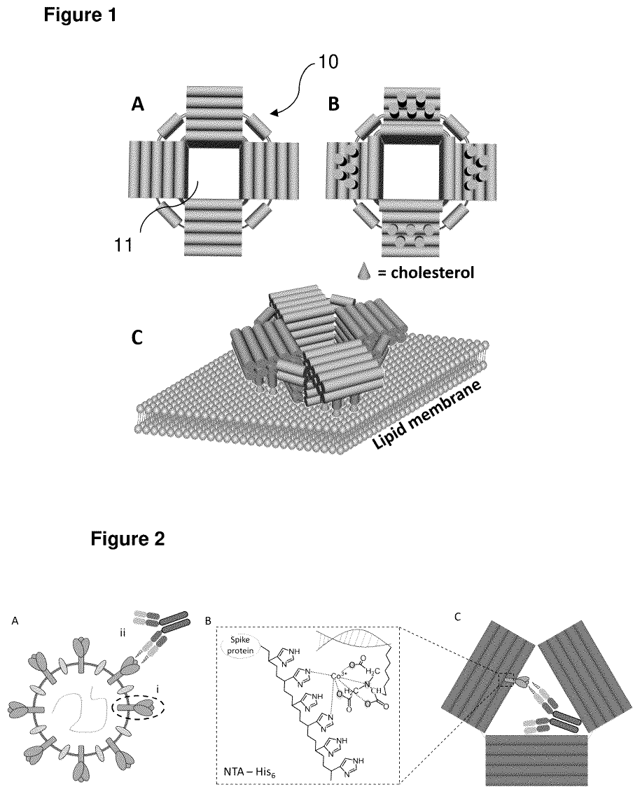

As shown in FIG. 1 (A)-(C), in an embodiment the sensing configuration comprises a nanostructure 10 that is embedded within a planar membrane or solid-state partition. The nanostructure is comprised of a nucleic acid nanopore having a lumen 11 the open ends of which define first and second apertures as described previously. The nanostructure inserts into and spans the membrane or solid-state partition with solutions on either side. On a first side of the membrane, that comes into contact with the first aperture, there is placed a sample fluid comprising an analyte that is to be tested for.

In a first embodiment, a signal readout is generated via measurement of an ionic electrical current that flows through the nanopore from the first side to a second side of the membrane, or vice versa, by way of a gradient of soluble ions present in the solution. The flow of this electrical current is measurable over a given period of time. It will be appreciated that alternative readouts may exist for identifying when an analyte is located optimally within the pore lumen. For example, alternative detection modalities based on field-effect transistors (FET), quantum tunnelling and optical methods such as fluorescence and plasmonic sensing may be utilised. For instance, a combination of a solid-state FET nanopore with an adjacent nanoribbon, nanotube, or nanowire, allows for sensing analyte molecules that interact with the lumen of the pore thereby disrupting the local electrical ionic current passing through the pore. In an alternative embodiment, transverse electrical measurements across the membrane of voltage, current or impedance may be made in order to generate a detectable signal readout upon binding of the analyte. It will be appreciated that whichever readout technology is adopted, the high fidelity of binding between the analyte and the correspondingly geometrically optimised nanostructure will improve the sensitivity of signal and reduce background noise.

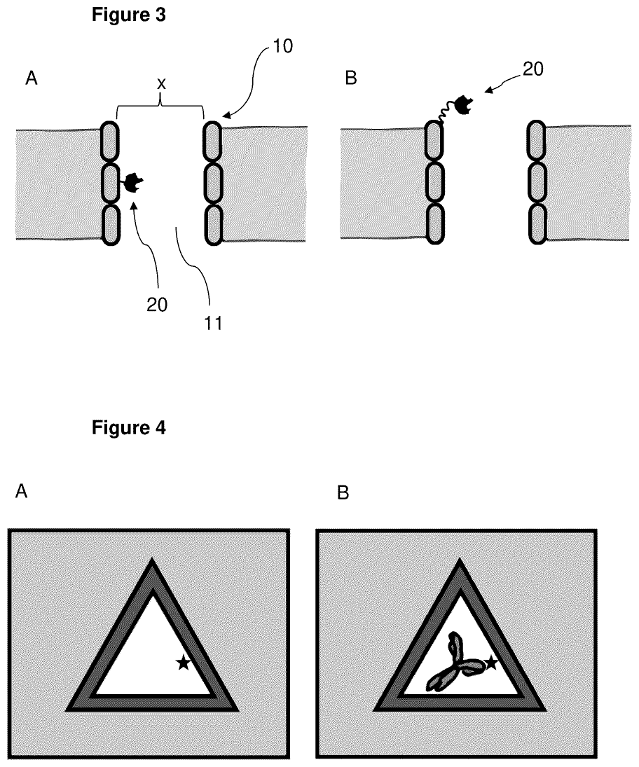

The nanostructure may further comprise one or more binding moieties which may be comprised of an affinity binding component or a molecule that is able to bind to an affinity binding molecule, such as an antigen. As shown in FIG. 3, a binding moiety 20 is suitably located proximate to the first aperture or within the lumen 11 (depicted by the region x in FIG. 3(A)). This facilitates binding to an analyte within the vicinity of the first aperture. In FIG. 3(B), an alternative embodiment is shown in which the binding moiety 20 is tethered to the nanopore at a location that allows the binding moiety to project fully or partially outside of the lumen 11. Upon binding of an analyte, the flow of electrical current through the first aperture, and therefore through the lumen of the nanostructure, is obstructed causing a blockade of all or a major portion of current passing through the nanopore. The level of signal readout—such as current blockade—can be measured in order to identify that the analyte has been detected. The one or more binding moieties may be attached to the pore via a covalent or non-covalent linkage, such as via avidin-biotin or His-tag type interaction.

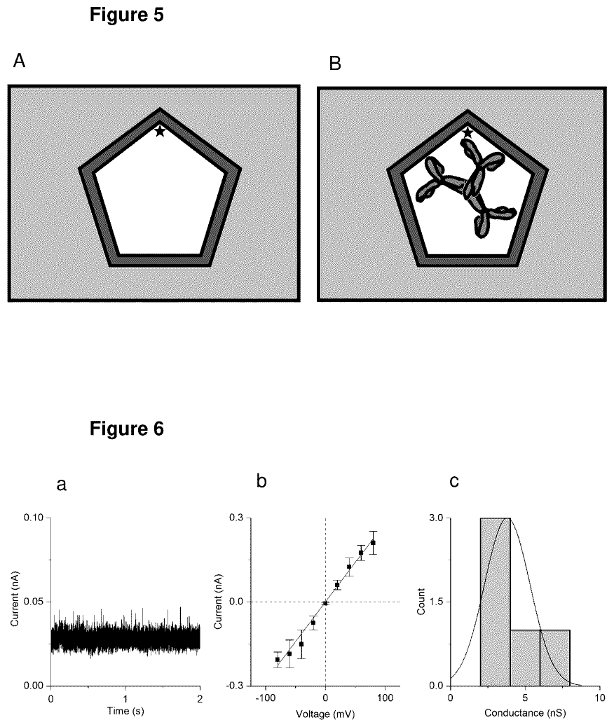

In accordance with the invention, a more effective signal is provided by optimising the geometry of the nanopore such that it accommodates and conforms to the shape of some part or a substantial portion of the three dimensional configuration of the analyte alone, or of the combination of the binding moiety-analyte complex. For example, a more effective current blockade or other electrical output signal can be generated. In FIG. 4(A), a triangular nanopore is depicted with a binding moiety attached to an internal wall of the lumen. In this instance the analyte is an IgG type antibody and the binding moiety may, thus, be an antigen that is recognised by the antibody (see FIG. 4(B)). In FIG. 5 (A), a pentagonal nanopore is shown with the binding moiety located at an internal junction/corner between adjacent walls of the nanopore. In FIG. 5 (B) the binding moiety is recognised and bound by a multidomain protein complex (for example, an IgM or IgA type polypeptide). Hence, the nanopore is configured so that it will receive the analyte into the lumen or at least into the region of the first aperture. This requires a correspondence of complementary geometry in terms of the tertiary structural confirmation of the nanopore lumen so as to allow for a high fidelity of interaction with the analyte. Binding of the analyte to the binding moiety is thereby facilitated by the geometry of the lumen. However, the nanopore does not in itself bind covalently, or irreversibly in any way to the analyte. Rather, the lumen and first aperture provide a suitable geometric support within which the affinity binding interaction between the binding moiety and the analyte may occur. This may be achieved through mild electrostatic interactions, Van der Waals forces or weak hydrogen bonding.

In an embodiment of the invention, the method of optimising the geometry of the nanopore enables the generation of nanopores substantially herein described.

A feature of the sensor nanopores as described herein is that the geometry of the lumen and/or aperture can be configured optimally to provide defined current readout upon analyte binding. A pre-defined shape complementarity, as expressed by the manner in which the nanopore receives the analyte, is one advantage that contributes to improved fidelity of readout and reduction of background signal ‘noise’. A further advantage provided by optimising the geometry of the nanopore lumen and/or aperture at the site of analyte binding is that it makes it possible to define a specific current blockade, such as a specific value or threshold reading, for a given analyte. This can assist in diagnostic or screening applications where multiple analytes may be present in a sample solution which could occupy the geometry of the pore with non-specific interactions but where only one class of analyte will bind with high specificity, thereby meeting the threshold reading. For instance, in high-throughput library screening of multiple compounds of similar origin, it is advantageous to configure the geometry of the nanopore such that it favours a specific binding interaction for the desired target analyte as identified by a current blockade that meets a specific threshold value. This level of analyte-specific optimisation is not currently possible for protein nanopores which have a predefined structure that is largely incapable of alteration.

An advantage of configuring the geometry of the nanopore such that it favours a specific binding interaction for the desired target analyte is that it further increases the information that can usefully be derived from a given analyte binding event. In addition to optimised electrical or other signal generation (e.g. current blockade), the fidelity of binding may also provide a specific detectable signature that is unique to a specific type of analyte. A signal signature may be useful where a plurality of closely related target analytes may be present, for example where closely related isoforms of an analyte are present, or where different multimeric forms of the analyte exist. In such cases a readout that provides a signature that reflects different binding events over time can be very valuable. In an alternative situation, other binding events may be optimised by selection of appropriate complementary pore geometry to allow for measurements of signal amplitude and/or signal duration (e.g. on/off time). Hence, the sensor nanopores as described herein facilitate a more diverse as well as optimised range of measurable sensor outputs that can provide better discrimination of analyte information to be obtained than has previously been considered possible.

A major challenge for identifying whether individuals have been exposed to an infectious disease in the past, such as COVID or HIV, involves identification of circulating antibodies within the plasma of that individual. In a specific embodiment of the invention shown in FIG. 2 (A-C) the binding moiety comprises a SARS-CoV-2 spike protein (i) which is immobilised within the lumen of a triangular shaped nucleic acid nanopore. The geometry of the nanopore is selected to favour binding of an IgG antibody analyte (ii), in particular, the pore defines a lumen that is an equilateral triangle with internal side lengths of around 20 nm. The nanopore may be embedded in a suitable membrane and exposed to a sample solution comprising the analyte. Binding of the analyte occurs within the lumen of the nanopore. Specificity of binding and detection via measurement of a current blockade, or other electrical or non-electrical signal readout, may be optimised by selection of the appropriate shape and dimensions of the nanopore. IgG antibodies are typically around 10 nm in size, hence selection of a nanopore lumen, aperture size and shape that is able to accommodate the antibody allows for optimal measured signal. In contrast, IgM antibody complexes are larger in size at around 35 nm and are of a different shape and configuration thereby requiring a nanopore with a different complementary geometry. In this way the configuration of the tertiary nanostructure in terms of dimensions, geometry and binding moiety placement (i.e. within or proximate to the lumen) may even influence factors such as the stereo- or regioselectivity of analyte binding. Size-exclusion by the nanopore size/shape, hence, improves the signal-to-noise ratio between long-binding target analyte interactions and short non-target analyte signals.

In a specific embodiment of the invention, the affinity binding molecule comprises a SARS-CoV-2 Spike receptor binding domain (RBD) or a fragment or derivative thereof.

In a specific embodiment of the invention, the analyte binding moiety comprises an affinity binding molecule that is tethered to the nanopore.

The affinity binding moiety may comprise a polynucleotide or a polypeptide that is capable of binding to an analyte present in a solution that surrounds the membrane-embedded nanopore. Where the binding molecule comprises a polypeptide or polynucleotide that is tethered to the nanopore, either within the lumen, an aperture or proximate to the cis or trans side of the nanopore, it may be selected from one of the group consisting of:
I. an enzyme—including a polymerase, a helicase, a gyrase, and a telomerase, as well as nucleic acid binding sub domains or derivatives thereof
II. synthetic or naturally derived affinity binding proteins and peptides—including affimers, antigen binding microproteins, engineered multiple repeat proteins, ankyrin binding domains, lactoferrins, cathelicidins, ficolins, collagenous lectins, T-cell receptor domains and defensins;
III. an antibody—including polyclonal, monoclonal, humanized and camelid antibodies, or antigen binding fragments and derivatives thereof, including Fab, scFv, Bis-scFv, VH, VL, V-NAR, VhH or any other antigen-binding single domain antibody fragment;
IV. synthetic or naturally derived affinity binding nucleic acids and nucleic acid analogues, including oligonucleotide probes, aptamers and ribozymes;
V. naturally occurring or synthetic small molecules, including drugs, fluorophores, metabolites and chemokines;
VI. an antigen or antigenic fragment; and
VII. signalling molecules and/or polypeptide receptors thereof, including binding domains of receptors, and receptor complexes.

Alternatively, the affinity binding molecule may comprise a small molecule, a lipid group, a polysaccharide group, a polymer or any other molecule naturally-occurring or synthetic molecule that is capable of effecting a specific affinity binding interaction with an analyte in solution. In an alternative embodiment, the nanostructure may lack any form of binding moiety and may rely on the geometrical optimisation of the pore or lumen to effect analyte binding. In this embodiment the nanostructure may comprise a substrate that includes a membrane or solid-state partition into which one or more nanostructures are embedded. The nanostructure is a nanopore that is typically configured so that it will receive the analyte into the lumen or at least into the region of the first aperture. This requires a correspondence of complementary geometry in terms of the tertiary structural conformation of the nanopore lumen so as to facilitate a high fidelity of interaction with the analyte. The nanopore lumen may be regularly or irregularly shaped to ensure close geometric correspondence with the tertiary structure of an analyte or a portion of an analyte, such as a domain or sub-unit thereof. Close correspondence may be defined as a gap between the periphery of the lumen and a surface of an analyte at any point about the lumen no more than 5 nm, 4 nm, 3 nm, 2 nm, 1 nm or 0.5 nm when the analyte is located, suitably in the geometrically optimal orientation, within the lumen of the nanopore. Alternatively, close correspondence may be defined as wherein at least >80%, >85%, >90%, >95% or up to 100% of the cross-sectional area of the lumen is occluded when the analyte is located, suitably in the geometrically optimal orientation, within the lumen of the nanopore. The lumen may be a regular or irregular polygon, such as a triangle, a quadrilateral (e.g. a square, a rectangle or a trapezoid), a pentagon, a hexagon, a heptagon, an octagon or a combination of such shapes. Alternatively or in addition, the channels may comprise components that are circular, elongate circular (elliptical) or elongate polygonal, such as rectangular, oblong or slot-shaped channels formed of 4 or more sides. By way of non-limiting example, a nanopore that is geometrically optimised to accommodate an IgG analyte may be configured to have a lumen that is generally triangular or even Y-shaped. Geometrical optimization may include configuring the nanostructure to receive all or a part of an analyte in a mating engagement, such as a lock and key type arrangement. It will be appreciated that optimizations of geometry to allow for detectable analyte engagement in the absence of a binding moiety may also be incorporated into embodiments described herein that include a binding moiety.

The nanopore structures of the present invention are suited to use in sensor applications that allow for the detection of a diverse range of potential analytes that may exist in a solution that is under test. Exemplary analytes may include:
peptides/polypeptides/proteins—folded, partially/completely unfolded;
enzymes;
protein/nucleic acid constructs;
molecules defined by size;
macromolecules within specified size ranges, for example, in ranges selected from: 1-10 kD, 1-50 kD, 1-100 kD, 10-50 kD, 10-100 kD, 20-50 kD, and 20-100 kD;
multi-protein complexes;
antigens or their antibodies;
glycoproteins;
carbohydrates;
biopolymers;
toxins;
metabolites and by products thereof;
cytokines;
nucleic acids.

The nanopore structures of the present invention may be incorporated within a plurality of improved devices and sensors. Such devices and sensors are useful in applications requiring to sensing and characterization of a variety of materials and analytes. By way of non-limiting example, particularly useful applications, including genome sequencing, protein sequencing, other biomolecular sequencing, and detection of ions, molecules, chemicals, small molecules, biomolecules, metal atoms, contaminants, polymers, nanoparticles etc. Such detecting and characterizing can, in turn, be used to diagnose diseases, in drug development, to identify contamination or adulteration or food or water supplies, and in quality control and standardization.

According to exemplary embodiments of the present invention as described, a sensor device typically comprises a substrate that includes a membrane or solid-state partition into which one or more nanopores are embedded. The substrate is placed to facilitate contact with a fluid (optionally an electrolytic solution) which comprises an analyte. At least one, and optionally a plurality of, device(s) are positioned relative to the substrate, wherein a given device generates a signal (e.g. mechanical, electrical, and/or optical) in response to detecting binding to and/or passage through the nanopore(s) of one or one or more analytes. The plurality of devices can be greater than 2 and as many as 100, or as many as 20, or as many as 10, or between 2 and 8 devices. Each device may be selected from one or more of the group consisting of: a field effect sensor; a plasmonic sensor; a laser based sensor; an interferometric sensor; a wave-guide sensor; a cantilever sensor; an acoustic sensor; a quartz crystal microbalance (QCM) sensor; an ultrasonic sensor; a mechanical sensor; a thermal sensor; an optical dye based sensor; a fluorimetric sensor; a calorimetric sensor; a luminometric sensor; a graphene sensor; a quantum dot sensor; a quantum-well sensor; a photoelectric sensor; a 2D material sensor; a nanotube or nanowire sensor; an enzymatic sensor; an electrochemical sensor, including a FET or BioFET sensor; a potentiometric sensor; a conductometric sensor; a capacitive sensors; and an electron-spin sensor. The devices may cooperate in the form of arrays allowing for multiplexed testing of multiple analytes. The sensor devices may further comprise special purpose hardware and systems (e.g., circuitry, processors, memory, GUIs etc.) that perform the specified functions or acts, or combinations of special purpose hardware and computer instructions, in order to render a functioning sensor device capable of providing a meaningful readout to a user.

In a particular embodiment of the invention, the device is portable. In an optional embodiment, the nanopores described herein are comprised within a flow cell. In a further optional embodiment of the invention, the nanopores described herein are embedded within a membrane comprised with a flow cell. In a specific embodiment of the invention, the nanopores described herein are embedded within a solid-state partition comprised within the flow cell. In a further specific embodiment, the devices described herein further comprise electrical measurement apparatus and/or fluorescence measurement apparatus.

Hence, according to embodiments of the invention suitably configured nanopore devices may enable a variety of different types of sensor measurements to be made. Typically, electrical measurements include: current measurements, impedance measurements, tunnelling measurements (Ivanov A P et al., Nano Lett. 2011 Jan. 12; 11(1):279-85), and FET measurements (International Application WO 2005/124888). Optical measurements may be combined with or based upon electrical measurements (Soni G V et al., Rev Sci Instrum. 2010 January; 81(1):014301), for instance, via conversion of ionic current into a fluorescent signal from an indicator dye (e.g. Fluo-8) arising from Ca²⁺ flux through a nanopore (Huang et al. Nat Nanotechnol. 2015 November; 10(11): 986-991). As mentioned previously, the measurement may be a transmembrane current or voltage measurement such as measurement of ionic current flowing through the nanopore. Alternatively, the signal may be obtained from measurement of a change in transverse membrane current, voltage and/or impedance value over time.

In alternative embodiments, methods for molecular sensing may comprise:
I. providing a sensor device as described herein;
II. contacting the nanopore with an analyte molecule and establishing a flow of ions through the at least one nanopore or an electron flow across the nanopore; and
III. measuring an electrical signal across the nanopore,
   wherein the molecular sensing comprises analyte molecule detection or characterisation, wherein a change in the electrical measurement is indicative of the analyte molecule.

In an optional embodiment of the present invention, the electrical measurement is selected from: a current measurement; an impedance measurement; a tunnelling measurement; and a field effect transistor (FET) measurement.

The invention is further illustrated by the following non-limiting examples.

### EXAMPLES

### Example 1—Analysis of Current Blockade in a Membrane Bound Triangular Pore Carrying a SARS CoV 2 Spike Protein in Testing for Analyte of an Anti-Spike Antibody in Solution

An equilateral triangular nucleic acid membrane-bound nanopore which defines a triangular lumen and, thus, a triangular aperture, was used as a test nanopore.

The pore was selected to have a 20 nm side length thereby providing a maximum internal width of around 17 nm. Typically IgG antibodies are around 10 nm in length and broadly Y shaped and so the pore was selected to have a geometry that corresponded to that of the analyte. The pore was constructed via DNA origami using an phiX174 scaffold and corresponding staple nucleic acid sequences and methods substantially as described in WO-2020/025974-A.

The phix174 scaffold sequence is provided in Table 1 below:

| | |
|---|---|
| phiX174 scaffold | GAGTTTTATCGCTTCCATGACGCAGAAGTTAACACTTTCGGATATTTCTGATGAGTCGA |
| (SEQ ID NO: 1) | AAAATTATCTTGATAAAGCAGGAATTACTACTGCTTGTTTACGAATTAAATCGAAGTGGA |
| | CTGCTGGCGGAAAATGAGAAAATTCGACCTATCCTTGCGCAGCTCGAGAAGCTCTTAC |
| | TTTGCGACCTTTCGCCATCAACTAACGATTCTGTCAAAAACTGACGCGTTGGATGAGGA |
| | GAAGTGGCTTAATATGCTTGGCACGTTCGTCAAGGACTGGTTTAGATATGAGTCACATT |
| | TTGTTCATGGTAGAGATTCTCTTGTTGACATTTTAAAAGAGCGTGGATTACTATCTGAGT |
| | CCGATGCTGTTCAACCACTAATAGGTAAGAAATCATGAGTCAAGTTACTGAACAATCCG |
| | TACGTTTCCAGACCGCTTTGGCCTCTATTAAGCTCATTCAGGCTTCTGCCGTTTTGGAT |
| | TTAACCGAAGATGATTTCGATTTTCTGACGAGTAACAAAGTTTGGATTGCTACTGACCG |
| | CTCTCGTGCTCGTCGCTGCGTTGAGGCTTGCGTTTATGGTACGCTGGACTTTGTAGGA |
| | TACCCTCGCTTTCCTGCTCCTGTTGAGTTTATTGCTGCCGTCATTGCTTATTATGTTCAT |
| | CCCGTCAACATTCAAACGGCCTGTCTCATCATGGAAGGCGCTGAATTTACGGAAAACAT |
| | TATTAATGGCGTCGAGCGTCCGGTTAAAGCCGCTGAATTGTTCGCGTTTACCTTGCGT |
| | GTACGCGCAGGAAACACTGACGTTCTTACTGACGCAGAAGAAAACGTGCGTCAAAAAT |
| | TACGTGCAGAAGGAGTGATGTAATGTCTAAAGGTAAAAAACGTTCTGGCGCTCGCCCT |
| | GGTCGTCCGCAGCCGTTGCGAGGTACTAAAGGCAAGCGTAAAGGCGCTCGTCTTTGG |
| | TATGTAGGTGGTCAACAATTTTAATTGCAGGGGCTTCGGCCCCTTACTTGAGGATAAAT |
| | TATGTCTAATATTCAAACTGGCGCCGAGCGTATGCCGCATGACCTTTCCCATCTTGGCT |
| | TCCTTGCTGGTCAGATTGGTCGTCTTATTACCATTTCAACTACTCCGGTTATCGCTGGC |
| | GACTCCTTCGAGATGGACGCCGTTGGCGCTCTCCGTCTTTCTCCATTGCGTCGTGGCC |
| | TTGCTATTGACTCTACTGTAGACATTTTTACTTTTTATGTCCCTCATCGTCACGTTTATGG |
| | TGAACAGTGGATTAAGTTCATGAAGGATGGTGTTAATGCCACTCCTCTCCCGACTGTTA |
| | ACACTACTGGTTATATTGACCATGCCGCTTTTCTTGGCACGATTAACCCTGATACCAATA |
| | AAATCCCTAAGCATTTGTTTCAGGGTTATTTGAATATCTATAACAACTATTTTAAAGCGC |
| | CGTGGATGCCTGACCGTACCGAGGCTAACCCTAATGAGCTTAATCAAGATGATGCTCG |
| | TTATGGTTTCCGTTGCTGCCATCTCAAAAACATTTGGACTGCTCCGCTTCCTCCTGAGA |
| | CTGAGCTTTCTCGCCAAATGACGACTTCTACCACATCTATTGACATTATGGGTCTGCAA |
| | GCTGCTTATGCTAATTTGCATACTGACCAAGAACGTGATTACTTCATGCAGCGTTACCA |
| | TGATGTTATTTCTTCATTTGGAGGTAAAACCTCTTATGACGCTGACAACCGTCCTTTACT |
| | TGTCATGCGCTCTAATCTCTGGGCATCTGGCTATGATGTTGATGGAACTGACCAAACGT |
| | CGTTAGGCCAGTTTTCTGGTCGTGTTCAACAGACCTATAAACATTCTGTGCCGCGTTTC |
| | TTTGTTCCTGAGCATGGCACTATGTTTACTCTTGCGCTTGTTCGTTTTCCGCCTACTGC |
| | GACTAAAGAGATTCAGTACCTTAACGCTAAAGGTGCTTTGACTTATACCGATATTGCTG |
| | GCGACCCTGTTTTGTATGGCAACTTGCCGCCGCGTGAAATTTCTATGAAGGATGTTTTC |
| | CGTTCTGGTGATTCGTCTAAGAAGTTTAAGATTGCTGAGGGTCAGTGGTATCGTTATGC |
| | GCCTTCGTATGTTTCTCCTGCTTATCACCTTCTTGAAGGCTTCCCATTCATTCAGGAACC |
| | GCCTTCTGGTGATTTGCAAGAACGCGTACTTATTCGCCACCATGATTATGACCAGTGTT |
| | TCCAGTCCGTTCAGTTGTTGCAGTGGAATAGTCAGGTTAAATTTAATGTGACCGTTTAT |
| | CGCAATCTGCCGACCACTCGCGATTCAATCATGACTTCGTGATAAAAGATTGAGTGTGA |
| | GGTTATAACGCCGAAGCGGTAAAAATTTTAATTTTTGCCGCTGAGGGGTTGACCAAGC |
| | GAAGCGCGGTAGGTTTTCTGCTTAGGAGTTTAATCATGTTTCAGACTTTTATTTCTCGCC |
| | ATAATTCAAACTTTTTTTCTGATAAGCTGGTTCTCACTTCTGTTACTCCAGCTTCTTCGG |
| | CACCTGTTTTACAGACACCTAAAGCTACATCGTCAACGTTATATTTTGATAGTTTGACGG |
| | TTAATGCTGGTAATGGTGGTTTTCTTCATTGCATTCAGATGGATACATCTGTCAACGCC |
| | GCTAATCAGGTTGTTTCTGTTGGTGCTGATATTGCTTTTGATGCCGACCCTAAATTTTTT |
| | GCCTGTTTGGTTCGCTTTGAGTCTTCTTCGGTTCCGACTACCCTCCCGACTGCCTATGA |
| | TGTTTATCCTTTGGATGGTCGCCATGATGGTGGTTATTATACCGTCAAGGACTGTGTGA |
| | CTATTGACGTCCTTCCCCGTACGCCGGGCAATAATGTTTATGTTGGTTTCATGGTTTGG |
| | TCTAACTTTACCGCTACTAAATGCCGCGGATTGGTTTCGCTGAATCAGGTTATTAAAGA |
| | GATTATTTGTCTCCAGCCACTTAAGTGAGGTGATTTATGTTTGGTGCTATTGCTGGCGG |
| | TATTGCTTCTGCTCTTGCTGGTGGCGCCATGTCTAAATTGTTTGGAGGCGGTCAAAAAG |
| | CCGCCTCCGGTGGCATTCAAGGTGATGTGCTTGCTACCGATAACAATACTGTAGGCAT |
| | GGGTGATGCTGGTATTAAATCTGCCATTCAAGGCTCTAATGTTCCTAACCCTGATGAGG |
| | CCGTCCCTAGTTTTGTTTCTGGTGCTATGGCTAAAGCTGGTAAAGGACTTCTTGAAGGT |
| | ACGTTGCAGGCTGGCACTTCTGCCGTTTCTGATAAGTTGCTTGATTTGGTTGGACTTGG |
| | TGGCAAGTCTGCCGCTGATAAAGGAAAGGATACTCGTGATTATCTTGCTGCTGCATTTC |
| | CTGAGCTTAATGCTTGGGAGCGTGCTGGTGCTGATGCTTCCTCTGCTGGTATGGTTGA |
| | CGCCGGATTTGAGAATCAAAAAGAGCTTACTAAAATGCAACTGGACAATCAGAAAGAGA |
| | TTGCCGAGATGCAAAATGAGACTCAAAAAGAGATTGCTGGCATTCAGTCGGCGACTTC |
| | ACGCCAGAATACGAAAGACCAGGTATATGCACAAAATGAGATGCTTGCTTATCAACAGA |
| | AGGAGTCTACTGCTCGCGTTGCGTCTATTATGGAAAACACCAATCTTTCCAAGCAACAG |
| | CAGGTTTCCGAGATTATGCGCCAAATGCTTACTCAAGCTCAAACGGCTGGTCAGTATTT |
| | TACCAATGACCAAATCAAAGAAATGACTCGCAAGGTTAGTGCTGAGGTTGACTTAGTTC |
| | ATCAGCAAACGCAGAATCAGCGGTATGGCTCTTCTCATATTGGCGCTACTGCAAAGGA |
| | TATTTCTAATGTCGTCACTGATGCTGCTTCTGGTGTGGTTGATATTTTTCATGGTATTGA |
| | TAAAGCTGTTGCCGATACTTGGAACAATTTCTGGAAAGACGGTAAAGCTGATGGTATTG |
| | GCTCTAATTTGTCTAGGAAATAACCGTCAGGATTGACACCCTCCCAATTGTATGTTTTCA |
| | TGCCTCCAAATCTTGGAGGCTTTTTTATGGTTCGTTCTTATTACCCTTCTGAATGTCACG |
| | CTGATTATTTTGACTTTGAGCGTATCGAGGCTCTTAAACCTGCTATTGAGGCTTGTGGC |
| | ATTTCTACTCTTTCTCAATCCCCAATGCTTGGCTTCCATAAGCAGATGGATAACCGCATC |
| | AAGCTCTTGGAAGAGATTCTGTCTTTTCGTATGCAGGGCGTTGAGTTCGATAATGGTGA |
| | TATGTATGTTGACGGCCATAAGGCTGCTTCTGACGTTCGTGATGAGTTTGTATCTGTTA |
| | CTGAGAAGTTAATGGATGAATTGGCACAATGCTACAATGTGCTCCCCCAACTTGATATT |
| | AATAACACTATAGACCACCGCCCCGAAGGGGACGAAAAATGGTTTTTAGAGAACGAGA |
| | AGACGGTTACGCAGTTTTGCCGCAAGCTGGCTGCTGAACGCCCTCTTAAGGATATTCG |
| | CGATGAGTATAATTACCCCAAAAAGAAAGGTATTAAGGATGAGTGTTCAAGATTGCTGG |
| | AGGCCTCCACTATGAAATCGCGTAGAGGCTTTGCTATTCAGCGTTTGATGAATGCAATG |
| | CGACAGGCTCATGCTGATGGTTGGTTTATCGTTTTTGACACTCTCACGTTGGCTGACGA |
| | CCGATTAGAGGCGTTTTATGATAATCCCAATGCTTTGCGTGACTATTTTCGTGATATTGG |
| | TCGTATGGTTCTTGCTGCCGAGGGTCGCAAGGCTAATGATTCACACGCCGACTGCTAT |
| | CAGTATTTTTGTGTGCCTGAGTATGGTACAGCTAATGGCCGTCTTCATTTCCATGCGGT |
| | GCACTTTATGCGGACACTTCCTACAGGTAGCGTTGACCCTAATTTTGGTCGTCGGGTAC |
| | GCAATCGCCGCCAGTTAAATAGCTTGCAAAATACGTGGCCTTATGGTTACAGTATGCCC |
| | ATCGCAGTTCGCTACACGCAGGACGCTTTTTCACGTTCTGGTTGGTTGTGGCCTGTTG |
| | ATGCTAAAGGTGAGCCGCTTAAAGCTACCAGTTATATGGCTGTTGGTTTCTATGTGGCT |
| | AAATACGTTAACAAAAAGTCAGATATGGACCTTGCTGCTAAAGGTCTAGGAGCTAAAGA |
| | ATGGAACAACTCACTAAAAACCAAGCTGTCGCTACTTCCCAAGAAGCTGTTCAGAATCA |
| | GAATGAGCCGCAACTTCGGGATGAAAATGCTCACAATGACAAATCTGTCCACGGAGTG |
| | CTTAATCCAACTTACCAAGCTGGGTTACGACGCGACGCCGTTCAACCAGATATTGAAG |
| | CAGAACGCAAAAAGAGAGATGAGATTGAGGCTGGGAAAAGTTACTGTAGCCGACGTTT |
| | TGGCGGCGCAACCTGTGACGACAAATCTGCTCAAATTTATGCGCGCTTCGATAAAAAT |
| | GATTGGCGTATCCAACCTGCA |

The staple sequences are provided in Table 2 below (SEQ ID Nos: 2 to 76):

| | |
|---|---|
| Tri-20-01 (SEQ ID | GTTGACCACCTACATACCAAATACGCTCCTGA |
| NO: 2) | |
| Tri-20-02 (SEQ ID | CGAGAGTCAGGTACGGCGGTCAGTAGTTTGTTACTCGTCAGAAAATCGAA |
| NO: 3) | |
| Tri-20-03 (SEQ ID | TGCGCTGGTAATAAGACGACCAGGAATT |
| NO: 4) | |
| Tri-20-04 (SEQ ID | GTTAATCGAGCGGCATGGTCAATATTAACACCAAACGTGA |
| NO: 5) | |
| Tri-20-05 (SEQ ID | TCTCCTCACTACCATGAACAAAATGTAATCCACGCTCTTT |
| NO: 6) | |
| Tri-20-06 (SEQ ID | GGAAGACGGCAGCAATAAACAGTAGTTGAAAGTACA |
| NO: 7) | |
| Tri-20-07 (SEQ ID | TAAAAAGTGAGAGCGCGAAGGAGTCAAGGAAG |
| NO: 8) | |
| Tri-20-08 (SEQ ID | TAAAATGTCAACAAACCAATCCAAGAGAATCTTCCAAC |
| NO: 9) | |
| Tri-20-09 (SEQ ID | ATCATCTTATTAGGGTTAGCCTCGGCATCCACGGCGCTTTGTATCAGG |
| NO: 10) | |
| Tri-20-10 (SEQ ID | GCGTCAGCGAAACGCAGTTTTTGACATATCCTACAAAGTCCAAGCA |
| NO: 11) | |
| Tri-20-11 (SEQ ID | GTGGAGCTTGCAGACCCATATCGTTCTCTAACCCAG |
| NO: 12) | |
| Tri-20-12 (SEQ ID | AGATGAAACCATTTTTGAATCTCTCAGA |
| NO: 13) | |
| Tri-20-13 (SEQ ID | CGGTCGTCAGAGTGTCAAAAACGATCATCAAAAGTGGAGG |
| NO: 14) | |
| Tri-20-14 (SEQ ID | ATAACTCCAAAGCAGCCTGAAACAAAGTCATTTGGCGAGAAAAGAT |
| NO: 15) | |
| Tri-20-15 (SEQ ID | TTGAACACAGAGGGCGCGGCAAAAGTGGTCTA |
| NO: 16) | |
| Tri-20-16 (SEQ ID | AGCAGCCTTATGGCCGTCAAGGAGCACATCGG |
| NO: 17) | |
| Tri-20-17 (SEQ ID | CAGAAGCCTGAATGAGGGAAACCATAACGAGCATTCAA |
| NO: 18) | |
| Tri-20-18 (SEQ ID | TTGAGTTGCGTAGCCATGGCAGCAACCTTAATAGAGGCCAAAGCGGTCTG |
| NO: 19) | |
| Tri-20-19 (SEQ ID | TTATAGATATCATCTTGATTAAGCGTTAAATCCAAAACGG |
| NO: 20) | |
| Tri-20-20 (SEQ ID | GAAACGTATCGGCGTGTGAATCATACCCTCGGCAGCAAGACCTCTAAT |
| NO: 21) | |
| Tri-20-21 (SEQ ID | GACTCATGATTTCTTAACCATACTCAGGCACACGAAAA |
| NO: 22) | |
| Tri-20-22 (SEQ ID | CCAACTGCGCCGCCAAGGGAAGTACGTG |
| NO: 23) | |
| Tri-20-23 (SEQ ID | TAGTCATGGAACCGCACGCAAAGCATGGTCAACGCTACCTGTCGAC |
| NO: 24) | |
| Tri-20-24 (SEQ ID | CAATATCACAAAAATACTGATAGCGATTGTTCAGTAACTT |
| NO: 25) | |
| Tri-20-25 (SEQ ID | GACTCAGAGACTCATATCTAAACCGAACGTGCCAAGCATAGAAAGGTC |
| NO: 26) | |
| Tri-20-26 (SEQ ID | GACAGATTTGTCATTGTGAGTGCGTTCTCTAC |
| NO: 27) | |
| Tri-20-27 (SEQ ID | GACCTTTAACTGGCGGCGATTCGTCACAGGTAGCCA |
| NO: 28) | |
| Tri-20-28 (SEQ ID | ATCAGAAAATACGCCACGCGCATAACTTTTCC |
| NO: 29) | |
| Tri-20-29 (SEQ ID | GACGGTTGACAGTCCCCATTAGCTGTCCTATTAGTGGTTGAACAGCATCG |
| NO: 30) | |
| Tri-20-30 (SEQ ID | GCAAAGTAGAGCTGCGCAAGGATACGATTTAATCAAGATA |
| NO: 31) | |
| Tri-20-31 (SEQ ID | AAGCATGTCAATGCTCAGTCTCTTTTtcatcttcacactact |
| NO: 32) | |
| Tri-20-32 (SEQ ID | TTACCTCCAAATGAAGAATAATTATtcatcttcacactact |
| NO: 33) | |
| Tri-20-33 (SEQ ID | GACGGGATACAATTCAGCTCGACGTTTTTGACtcatcttcacactact |
| NO: 34) | |
| Tri-20-34 (SEQ ID | GGCGCTGCGTAACCGTCTTCCTCTACGCAATAtcatcttcacactact |
| NO: 35) | |
| Tri-20-35 (SEQ ID | TAAGGCCAAAGCGGCTAGGCCACAGCAAGGTCtcatcttcacactact |
| NO: 36) | |
| Tri-20-36 (SEQ ID | CAATGGCGCCAGGCAATGGAtcatcttcacactact |
| NO: 37) | |
| Tri-20-37 (SEQ ID | CCAGCGCCAGCGATAACCGGATCCACTGCTACtcatcttcacactact |
| NO: 38) | |
| Tri-20-38 (SEQ ID | TTAAGCGAGCGCCAGAACGTTTTTTACCGAACGTCACTCAAGTAtcatcttcacactact |
| NO: 39) | |
| Tri-20-39 (SEQ ID | ACTCGCGACAGCTTGGTTTTTAGTGAGTTTTAGCCATAACCCAGtcatcttcacactact |
| NO: 40) | |
| Tri-20-40 (SEQ ID | TATCGTGTTTCCCGCAAGGTAAACGCGAGAACATAACCATtcatcttcacactact |
| NO: 41) | |
| Tri-20-41 (SEQ ID | CTTATCAACAGGGCGTACCAGAGTCAtcatcttcacactact |
| NO: 42) | |
| Tri-20-42 (SEQ ID | ACGTTCCAAGAGCTCAAAGAAACGCGGCCATCAACACTCAGTAAtcatcttcacactact |
| NO: 43) | |
| Tri-20-43 (SEQ ID | AGTAAATTTGAGCAGATTTGAGTAATTCAGTGtcatcttcacactact |
| NO: 44) | |
| Tri-20-44 (SEQ ID | TTAATAATGTTTTCCGTAAAGGCCAtcatcttcacactact |
| NO: 45) | |
| Tri-20-45 (SEQ ID | ACTTTCATAGCCAGATTAGAGCGCATGAGCAAATTAAACGtcatcttcacactact |
| NO: 46) | |
| Tri-20-46 (SEQ ID | TCGGGCTTCAATAAACTCTGtcatcttcacactact |
| NO: 47) | |
| Tri-20-47 (SEQ ID | TTAACGTATGTTCCATCTTTAGCACTTGGTAAGTTGGATTTCGCtcatcttcacactact |
| NO: 48) | |
| Tri-20-48 (SEQ ID | GTCAGTTCACAGAATGACACGACCCAGATACAAACTCATCATTAtcatcttcacactact |
| NO: 49) | |
| Tri-20-49 (SEQ ID | GCTAAAAATTAGGAGCCTGTCGCATTGCAACCAACCCGATGGGCtcatcttcacactact |
| NO: 50) | |
| Tri-20-50 (SEQ ID | GTCAGTAATTTAGACAGCACGTAAAGGGGCCGAAGCCCCTAATTtcatcttcacactact |
| NO: 51) | |
| Tri-20-51 (SEQ ID | CAATAGCGAGGGTCCGCCAGCAGTCCACTCGAATTTAGACAGGCtcatcttcacactact |
| NO: 52) | |
| Tri-20-52 (SEQ ID | CCCTTTGTAGCAATCGCGAAtcatcttcacactact |
| NO: 53) | |
| Tri-20-53 (SEQ ID | AAGCTAGAAGTCGTCGGGAGAGGAGTGGACCAGTAGATGAAGTAtcatcttcacactact |
| NO: 54) | |
| Tri-20-54 (SEQ ID | CAGTTGCGTACCAGGAAGTGCTTCTGtcatcttcacactact |
| NO: 55) | |
| Tri-20-55 (SEQ ID | AACCAGAACGTGAAAAAGTGGAAGCtcatcttcacactact |
| NO: 56) | |
| Tri-20-56 (SEQ ID | GTCAGTATCAAGTAAACATAAGAGAGAAAACTtcatcttcacactact |
| NO: 57) | |
| Tri-20-57 (SEQ ID | GTCGCATAGAAATATAACTGGTAGCTTTCGTATTTTGCGAtcatcttcacactact |
| NO: 58) | |
| Tri-20-58 (SEQ ID | cctacgagtcagtcctTGGGGATAACATCATGGTGCAT |
| NO: 59) | |
| Tri-20-59 (SEQ ID | cctacgagtcagtcctCCTTTCAACGTGAGCCAGGAGGAAGCGGATGTTT |

NO: 60)

| | |
|---|---|
| Tri-20-60 (SEQ ID | cctacgagtcagtcctGATAATCTGGTTGAACGGCGAAGC |
| NO: 61) | |
| Tri-20-61 (SEQ ID | cctacgagtcagtcctACTCTTGTGCCAATTCATCCACGA |
| NO: 62) | |
| Tri-20-62 (SEQ ID | cctacgagtcagtcctAGTATAAAGAAATGCCAACGCAAGCCTCCGAGCA |
| NO: 63) | |
| Tri-20-63 (SEQ ID | cctacgagtcagtcctTTAATCCTTCTCAGAGCGCATAAAGTGCAATGAA |
| NO: 64) | |
| Tri-20-64 (SEQ ID | cctacgagtcagtcctAAGTTGGGCATACATACAACGCCCTGCATACGAAAAGACACGTC |
| NO: 65) | |
| Tri-20-65 (SEQ ID | cctacgagtcagtcctCATGCGGCAGACGAGCTTAGTACCTCGCAACGGCTGCGGACGAC |
| NO: 66) | |
| Tri-20-66 (SEQ ID | cctacgagtcagtcctCGACTTTGAATATTAGACATGCAA |
| NO: 67) | |
| Tri-20-67 (SEQ ID | cctacgagtcagtcctTCTCTTTTCATTTTCACATTCTGATTCTGAACAGCTTCTTAACG |
| NO: 68) | |
| Tri-20-68 (SEQ ID | cctacgagtcagtcctACTGATCAGCATTGGGATTATCATAAAACATACGAC |
| NO: 69) | |
| Tri-20-69 (SEQ ID | cctacgagtcagtcctCGTCACGTCCTGCGTGTAGCAA |
| NO: 70) | |
| Tri-20-70 (SEQ ID | cctacgagtcagtcctTATCCTTATCATCCTTGATTTCATCGCTGAATAGCA |
| NO: 71) | |
| Tri-20-71 (SEQ ID | cctacgagtcagtcctGATGTCTCATTTGAATCGTTAGTTGATGAAGCCACT |
| NO: 72) | |
| Tri-20-72 (SEQ ID | cctacgagtcagtcctCAGGTTGGTATCCGAACTGCTTTATTCGTAAACAAG |
| NO: 73) | |
| Tri-20-73 (SEQ ID | cctacgagtcagtcctCTGCTGTTAACATGCTTAGGGATTTTATAATAGTTG |
| NO: 74) | |
| Tri-20-74 (SEQ ID | cctacgagtcagtcctGAAAGACGAAAAATGTTTCACCATATCCTTCATGAA |
| NO: 75) | |
| Tri-20-75 (SEQ ID | cctacgagtcagtcctATAGCAATTCAGCGCCTTTAAG |
| NO: 76) | |

The analyte binding molecule is linked to the nanopore via a nitrilotriacetate (NTA) conjugation approach. Oligo-DNA can be modified with NTA, which has high affinity to a His-tag linked to a recombinant protein (for example a Covid spike protein) via the complexation of Ni²⁺ or Co³⁺ (Shimada et al. (2008) Biotechnology Letters volume 30, pages 2001-2006). The sequences used to enable the linkage of the binding moiety to the nanopore via an NTA modified DNA sequence that hybridises to part of a staple linker sequence are shown in Table 3 below.

| | |
|---|---|
| Tri-20-NTA-DNA | GTCGGAAGCATGCAACACAGTTGTGGAGCTTGCAGACCCATATCGTTCTCTAACCCAG |
| binding (SEQ ID | |
| NO: 77) | |
| NTA-modified DNA | [NTA] TTTCTGTGTTGCATGCTTCCGAC |
| (SEQ ID NO: 78) | |

The human SARS-CoV-2 antibodies used as analyte were from Antibodies-online (ABIN6952547). The SARS-CoV-2 Spike RBD (Receptor-binding domain, 45 pmol) used as binding protein was from the same company (ABIN6952627).

To prepare the nucleic acid sensor pore (Tri-20-Spike pore), carrying a SARS-CoV-2 Spike protein, the NTA-modified DNA oligonucleotide (30 pmol) was mixed with the His-tagged of the SARS-CoV-2 Spike1 protein in HEPES buffer (25 mM, pH 7.6) containing CoCl₂ (20 μM), NaCl (400 mM) and Tween-20 (0.02 v/v %) and incubated for 1 hour at RT. The solution was treated with H₂O₂ (20 mM) for 1 hour. The DNA-protein conjugate was then examined by 10% PAGE analysis. The freshly prepared DNA-protein conjugate was then mixed with purified Tri-20 bearing the complementary binding strand for the DNA-protein conjugate at a ratio of 1.5:1 and incubated for 1 hour at RT. The Tri-20-Spike pore was aliquoted and stored in −20° C. for further use.

Single-channel current recordings were carried out using an integrated chip-based, parallel recording setup (Orbit Mini and Orbit 16, Nanion Technologies, Munich, Germany) with multielectrode-cavity-array (MECA) chips (IONERA, Freiburg, Germany). Bilayers were formed of 1,2-Diphytanoyl-sn-Glycero-3-Phosphatidylcholine (DPhPC) dissolved in octane to a final concentration of 10 mg/mL. Electrophysiological buffer was composed of 1M KCl, 10 mM HEPES, pH 7.4. For pore insertions, Tri-20-Spike pores were mixed in a 2:1 (vol/vol) ratio with 0.5% OPOE (n-octyloligooxyethylene) in 1 M KCl, 10 mM HEPES, pH 7.4. The mixture was applied to the cis chamber and insertions monitored by increases in conductance steps. The current traces were not Bessel-filtered and acquired at 10 KHz using Element Data Recorder software (Element s.r.l., Italy).

For protein sensing experiments, anti-spike antibody (the analyte) was diluted in electrophysiological buffer to the desired concentration. Upon successful nanopore insertion, diluted protein was added to the cis chamber. The Orbit 16 was used for protein sensing experiments; the Orbit mini was used in all other electrophysiological experiments. The Orbit 16 and Orbit mini are grounded at the cis and trans, respectively. To aid comparison, voltages were normalized and are presented as positive in relation to the cis chamber. Single-channel analysis was performed using Clampfit software (Molecular Devices, Sunnyvale, CA, USA).

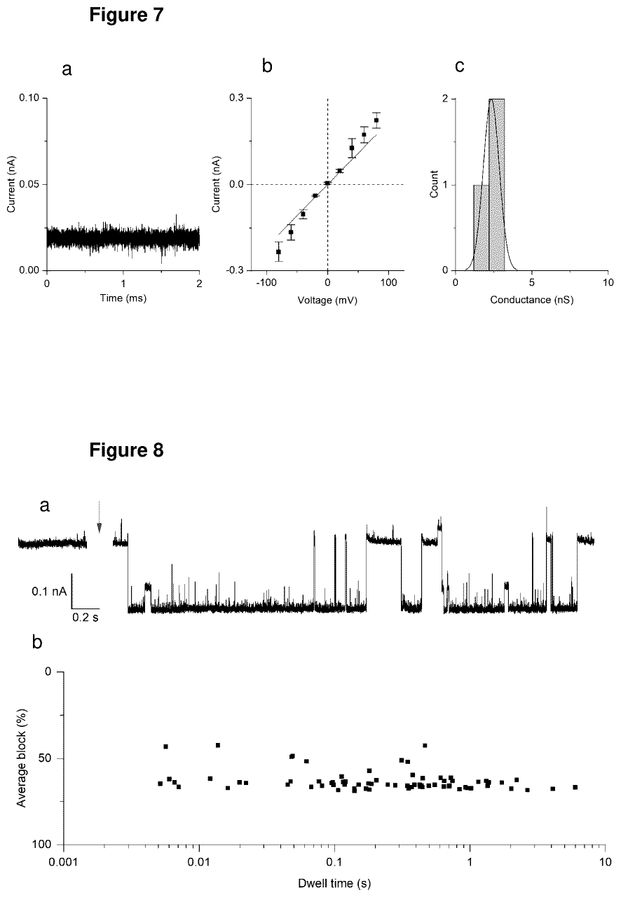

A plain un-functionalised triangular nanopore was used to establish baseline current flow measurements, current-voltage curve, and conductance histogram as shown in FIG. 6 (a-c). In FIG. 7 (a-c) current flow results, current voltage curve and conductance histogram are shown for a triangular nanopore functionalised with SARS CoV 2 spike protein (Tri-20-Spike pore) modified by addition of an N-terminal His6-tag as a binding moiety attached to the nanopore via a Co³⁺ metal chelate bridge (see FIG. 2B). As expected, the current flow through the functionalised pore is lower as the spike protein occludes part of the pore lumen. The presence of anti-spike antibody as analyte is detected rapidly at nanomolar concentrations as shown in FIG. 8 (a), where a rapid drop in current flow, termed a ‘blockade’, represents the binding of an individual antibody to the DNA nanopore. The binding events are summarised with a scatter plot (FIG. 8(b)) where each binding event is represented as a dot defined by the duration of the blockade and the current magnitude of the blockade.

The analysis of the current flow recording via the scatter plot shows that antibody binding to the pore led to a current blockade events with an average of 63.2±6.0% reduction of current compared to the open pore current. The duration of the blockade events was 0.61±0.35 s obtained from the exponential decay fits of the dwell time histogram. The data demonstrate that binding of antibodies to the sensor pore lead to defined current blockades.

### Example 2—Analysis of Membrane Bound Triangular Pore Carrying a SARS CoV 2 Spike Protein Using a MinION Device

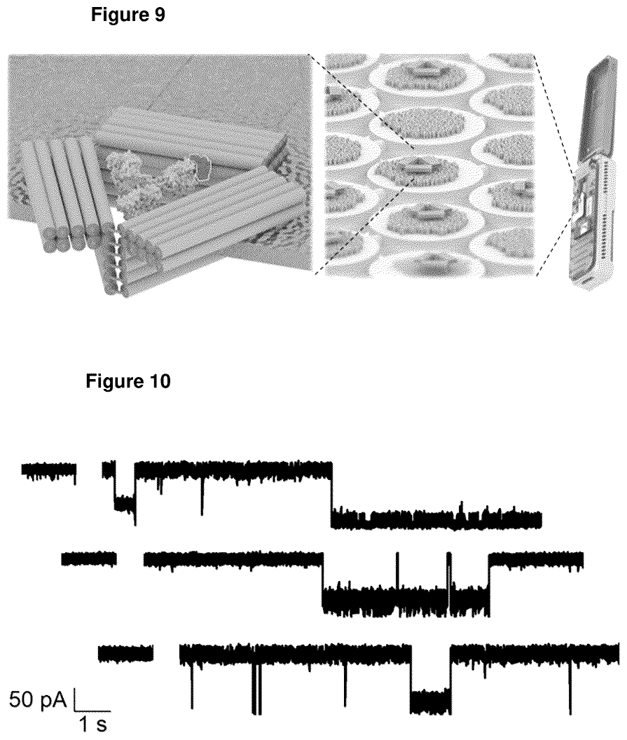

The capacity for nanopores to insert into membranes comprised within high-throughput MinION devices to rapidly sense human SARS-CoV-2 antibodies was evaluated. To achieve a better geometric match to the Y-shaped analyte, the triangular pore Tri-20 was selected (FIG. 9).

To fold the Tri-20 pore, the phix174 scaffold was first mixed at a 1:10 ratio with corresponding staples in 0.5×TAE (20 mM Tris base, 10 mM acetic acid, 0.5 mM EDTA, pH 8.3) supplemented with 16 mM MgCl₂. The DNA origami structures were folded using a 40-hour folding program: firstly, the solutions were heated at 75° C. for 10 min to denature undesired DNA secondary structures; then, for annealing, the solutions were cooled from 65° C. to 25° C. at a rate of 1ºC per hour, followed by cooling to 10° C. at a rate of 1° C. per 5 min, and kept at 4° C. until collection. After the folding process, the DNA origami structures were purified by excision from 1% agarose gel in 0.5×TBE buffer (45 mM Tris-borate, 1 mM EDTA, pH 8.3) supplemented with 11 mM MgCl₂. The cholesterol-tagged pores were prepared by adding cholesterol-labelled DNA oligonucleotides to the purified DNA pores at a stoichiometry of 1.5 relative to the total number of DNA cholesterol attachment sites at the DNA pores. The cholesterol-tagged pores were freshly prepared and used for the membrane binding and current recordings experiments on the same day.

To prepare the nucleic acid sensor pore (Tri-20-Spike pore), carrying a SARS-CoV-2 Spike protein, the NTA-modified DNA oligonucleotide (30 pmol) was mixed with the His-tagged receptor binding domain (45 μmol) of the SARS-CoV-2 Spike1 protein in HEPES buffer (25 mM, pH 7.6) containing CoCl₂ (20 μM), NaCl (400 mM) and Tween-20 (0.02 v/v %) and incubated for 1 hour at RT. The solution was treated with H₂O₂ (20 mM) for 1 hour. The DNA-protein conjugate was then examined by 10% PAGE analysis. The freshly prepared DNA-protein conjugate was then mixed with purified Tri-20 bearing the complementary binding strand for the DNA-protein conjugate at a ratio of 1.5:1 and incubated for 1 hour at RT. The Tri-20-Spike pore was aliquoted and stored in −20° C. for further use.

Single-channel current recordings were carried out using an integrated chip-based, parallel recording setup (Orbit Mini and Orbit 16, Nanion Technologies, Munich, Germany) with multielectrode-cavity-array (MECA) chips (IONERA, Freiburg, Germany). Bilayers were formed of 1,2-Diphytanoyl-sn-Glycero-3-Phosphatidylcholine (DPhPC) dissolved in octane to a final concentration of 10 mg/mL. Electrophysiological buffer was composed of 1 M KCl, 10 mM HEPES, pH 7.4. For pore insertions, Tri-20-Spike pores were mixed in a 2:1 (vol/vol) ratio with 0.5% OPOE (n-octyloligooxyethylene) in 1 M KCl, 10 mM HEPES, pH 7.4. The mixture was applied to the cis chamber and insertions monitored by increases in conductance steps. The current traces were not Bessel-filtered and acquired at 10 KHz using Element Data Recorder software (Element s.r.l., Italy).

For protein sensing experiments, anti-spike antibody (the analyte) was diluted in electrophysiological buffer to the desired concentration. Upon successful nanopore insertion, diluted protein was added to the cis chamber. The Orbit 16 was used for protein sensing experiments; the Orbit mini was used in other electrophysiological experiments. The Orbit 16 and Orbit mini are grounded at the cis and trans, respectively. To aid comparison, voltages were normalized and are presented as positive in relation to the cis chamber. Single-channel analysis was performed using Clampfit software (Molecular Devices, Sunnyvale, CA, USA).

Electrophysiological current recordings were also conducted using the MinION flow cell-based analysis device (Oxford Nanopore Technologies, Oxford, UK). The electrolyte solution was 1 M KCl equivalent. Proprietary membranes of the Oxford Nanopore Technologies were pre-formed on the flow cell. POPC SUVs were used to facilitate the fusion of DNA nanopore to the MinION membranes. The vesicles were prepared by drying a POPC solution (20 mg/mL in chloroform, 50 μL) in a glass vial (2 mL) by argon airflow, resuspension of the dried film in 1× incubation buffer (0.5×TAE with 500 mM NaCl to 1 mL) and sonication for 30 min. For pore insertion, DNA nanopores and vesicles at a ratio of 3 nM:1 mM lipid were incubated at 4° C. overnight. Subsequently, DNA nanopore containing vesicles were added to the MinION flow cells, 20 μl at a time. To promote fusion with the planar membranes, a voltage ramp protocol from 50 mV to 300 mV was applied. Upon successful insertions, recordings for conductance and IV curves were acquired. For molecular sensing with the Tri-20-Spike nanopore, human SARS-CoV-2 antibodies (Antibodies-online, ABIN6952547) was added directly to the flow cell.

Tri-20 without the antibody receptor inserted successfully into MinION membrane arrays (FIG. 9) and yielded steady single-channel currents with a mean conductance at 7.83±0.71 nS (SEM, n=9)(FIG. 12a).

Tri-20 was turned into a sensor for SARS-CoV-2 antibodies by specifically attaching the cognate receptor, SARS-CoV-2 Spike protein, into the pore lumen (FIG. 9). The resulting Tri-20-Spike pore yielded stable read-out traces in highly parallel MinION recordings (FIG. 12(b)). The average conductance of Tri-20-Spike pores was 1.8±0.2 nS (SEM, n=23)(FIG. 12(b)), which was lower than for Tri-20 (FIG. 12(a)), and confirms the presence of the Spike protein in the lumen of the nanopore.

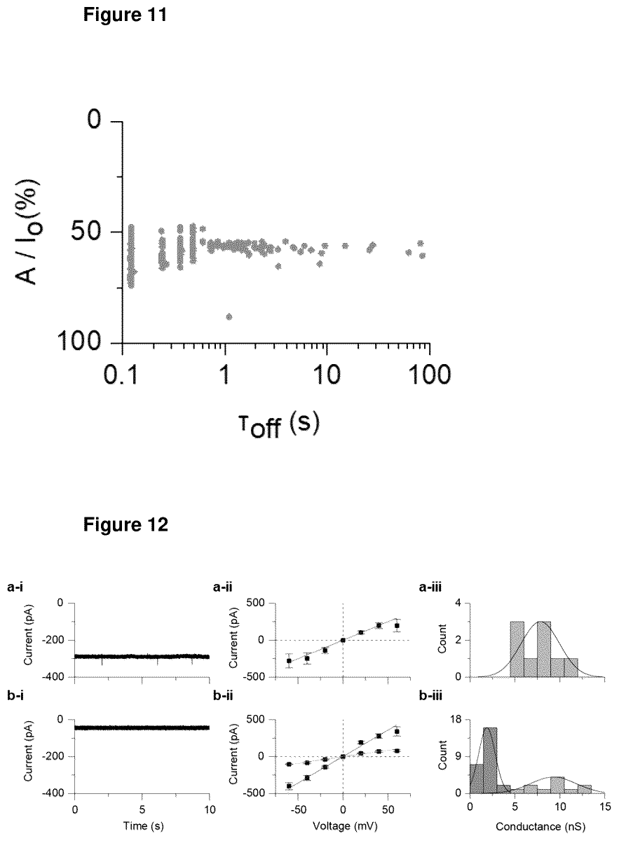

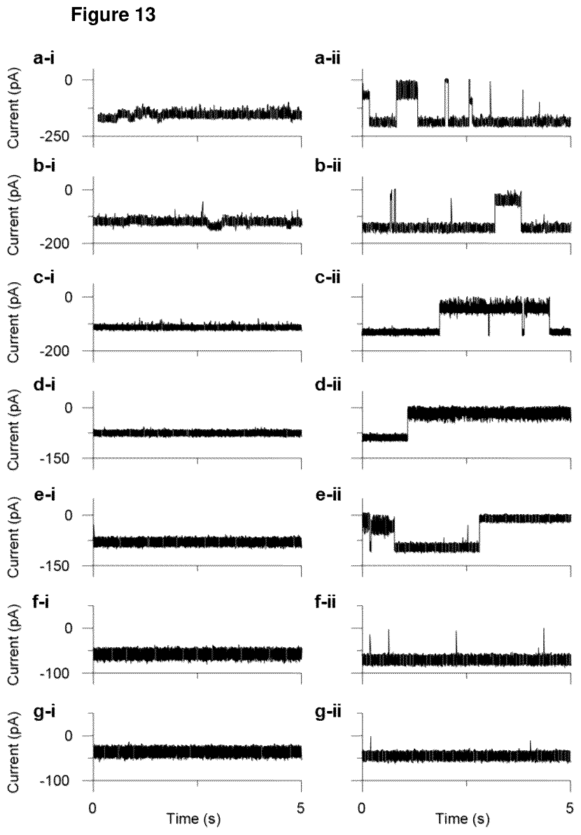

Addition of human SARS-CoV-2 antibodies to Tri-20-Spike led to current blockades (FIG. 10, FIG. 13) which represent individual binding events to the Spike receptor. The binding events had a relative amplitude of 61.7±6.9% (±STD, n=357) and an average dwell time of 1.7±8.6 s (±STD, n=357) (FIG. 11), implying that the triangular pores' shape which matched the Y-shaped analyte led to a high-blockade read-out.

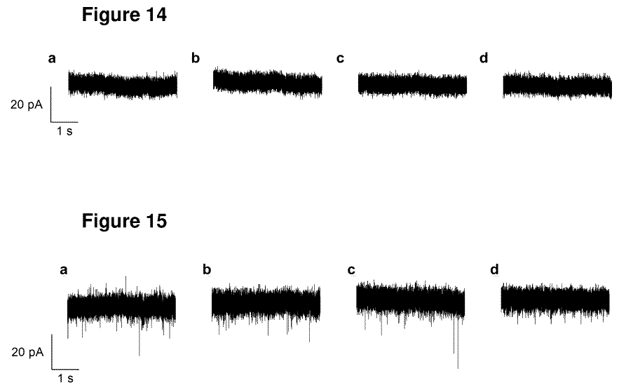

Antibody binding was found to be specific as anti-SARS-CoV-2 antibody did not elicit blockades in Tri-20-link (pore plus linker, without spike protein) (FIG. 14) and neither did addition of a non-specific antibody (FIG. 15).

Although particular embodiments of the invention have been disclosed herein in detail, this has been done by way of example and for the purposes of illustration only. The aforementioned embodiments are not intended to be limiting with respect to the scope of the appended claims, which follow. The choice of nucleic acid starting material, the clone of interest, or type of library used is believed to be a routine matter for the person of skill in the art with knowledge of the presently described embodiments. It is contemplated by the inventors that various substitutions, alterations, and modifications may be made to the invention without departing from the spirit and scope of the invention as defined by the claims.

1-23. (canceled)

24. A sensing nucleic acid nanopore comprising:
a nucleic acid nanopore, wherein the nanopore defines a central lumen passing therethrough, and wherein the central lumen has a cis-facing first open end that defines a first aperture and a trans-facing second open end that defines a second aperture; and
an analyte binding moiety, wherein the analyte binding moiety comprises an affinity binding molecule, and wherein the analyte binding moiety is located within the central lumen between the first and second apertures;
wherein the nanopore has a tertiary structure and the tertiary structure of the nanopore is configured to conform to a three-dimensional shape of at least a portion of an analyte molecule.

25. The nanopore of claim 24, wherein the nanopore is configured to be located within a membrane.

26. The nanopore of claim 25, wherein the nanopore further comprises at least one hydrophobic anchor that facilitates insertion of the nanopore into the membrane.

27. The nanopore of claim 26, wherein the at least one hydrophobic anchor is comprised of a polynucleotide strand and at least one hydrophobic anchor molecule.

28. The nanopore of claim 27, wherein the at least one hydrophobic anchor molecules are:
a. attached to and spaced substantially equidistantly about the periphery of the nanopore, and wherein the at least one hydrophobic anchor molecules are orientated radially outwardly from the longitudinal axis of the central lumen of the nanopore; and/or
b. attached to a membrane-facing side of the nanopore or a portion thereof such that the at least one hydrophobic anchor molecules are orientated to interact with and/or extend perpendicularly into the membrane once inserted.

29. The nanopore of claim 28, wherein the nanopore comprises at least four hydrophobic anchor molecules.

30. The nanopore of claim 29, wherein the at least one hydrophobic anchor molecule is selected from the group consisting of: a lipid; and a porphyrin.

31. (canceled)

32. The nanopore of claim 30, wherein:
the sterols are selected from the group consisting of: cholesterol; derivatives of cholesterol; phytosterol; ergosterol; and bile acid;
the alkylated phenols are selected from the group consisting of: methylated phenols; dolichols and tocopherols;
the flavones are selected from the group consisting of: flavanone containing compounds; and 6-hydroxyflavone;
the saturated and unsaturated fatty acids are selected from the group consisting of: derivatives of lauric acid; oleic acid; linoleic acid; and palmitic acids; and/or
the synthetic lipid molecule is dodecyl-beta-D-glucoside.

33. The nanopore of claim 32, wherein the analyte binding moiety is located proximate to or within the first aperture.

34. The nanopore of claim 33, wherein the affinity binding molecule comprises a molecule selected from the group consisting of:
I. an enzyme—including a polymerase, a helicase, a gyrase, and a telomerase, as well as nucleic acid binding sub domains or derivatives thereof;
II. synthetic or naturally derived affinity binding proteins and peptides—including affimers, antigen binding microproteins, engineered multiple repeat proteins, ankyrin binding domains, lactoferrins, cathelicidins, ficolins, collagenous lectins, T-cell receptor domains and defensins;
III. an antibody—including polyclonal, monoclonal, humanized and camelid antibodies, or antigen binding fragments and derivatives thereof, including Fab, scFv, Bis-scFv, VH, VL, V-NAR, VhH or any other antigen-binding single domain antibody fragment;
IV. synthetic or naturally derived affinity binding nucleic acids and nucleic acid analogues, including oligonucleotide probes, aptamers and ribozymes;
V. naturally occurring or synthetic small molecules, including drugs, fluorophores, metabolites and chemokines;
VI. an antigen or antigenic fragment; and
VII. signalling molecules and/or polypeptide receptors thereof, including binding domains of receptors, and receptor complexes.

35. The nanopore of claim 34, wherein the affinity binding molecule comprises a SARS-CoV-2 Spike receptor binding domain (RBD) or a fragment or derivative thereof.

36. The nanopore of claim 35, wherein the analyte binding moiety comprises the affinity binding molecule that is tethered to the nanopore.

37. The nanopore of claim 36, wherein the tertiary structure of the nanopore is configured to conform to a three-dimensional shape of at least a portion of the analyte molecule to facilitate a mating engagement between the analyte molecule and the nanopore.

38. The nanopore of claim 37, wherein the tertiary structure of the nanopore is configured to facilitate a stereo- or regio-specific interaction between a binding moiety and the analyte molecule.

39. The nanopore of claim 38, wherein the analyte molecule is selected from the list consisting of: a peptide; a polypeptide; a protein; a glycoprotein; an enzyme; a nucleic acid; an oligonucleotide; a polynucleotide; a protein-nucleic acid complex; a multi-protein complex; an antigen; an antibody; a macromolecule within the size range: 1-10 kD, 1-50 kD, 1-100 kD, 10-50 kD, 10-100 kD, 20-50 kD, and 20-100 kD; a carbohydrate; a biopolymer; a toxin; a metabolite; and/or a cytokine.

40. The nanopore of claim 39, wherein the first aperture and/or central lumen has a regular polygonal shape.

41. The nanopore of claim 40, wherein the regular polygonal shape is selected from the group consisting of: a triangle; a square; a quadrilateral; a pentagon; a hexagon; a heptagon; and an octagon.

42. The nanopore of claim 41, wherein the first aperture and/or central lumen has an irregular polygonal shape.

43. The nanopore of claim 42, wherein assembly of the nanopore and/or components thereof is via DNA origami techniques.

44. A membrane into which is inserted at least one nanopore as described in claim 43.

45-83. (canceled)


SEQUENCE LISTING (78 sequences)

SEQ ID NO: 1 - DNA, 5386 bp, Bacteriophage phi-X174
gagttttatcgcttccatgacgcagaagttaacactttcggatatttctgatgagtcgaaaaattatcttgataaagcaggaattactactgcttgtttacgaattaaatcgaagtggactgctggcggaaaatgagaaaattcgacctatccttgcgcagctcgagaagctcttactttgcgacctttcgccatcaactaacgattctgtcaaaaactgacgcgttggatgaggagaagtggcttaatatgcttggcacgttcgtcaaggactggtttagatatgagtcacattttgttcatggtagagattctcttgttgacattttaaaagagcgtggattactatctgagtccgatgctgttcaaccactaataggtaagaaatcatgagtcaagttactgaacaatccgtacgtttccagaccgctttggcctctattaagctcattcaggcttctgccgttttggatttaaccgaagatgatttcgattttctgacgagtaacaaagtttggattgctactgaccgctctcgtgctcgtcgctgcgttgaggcttgcgtttatggtacgctggactttgtaggataccctcgctttcctgctcctgttgagtttattgctgccgtcattgcttattatgttcatcccgtcaacattcaaacggcctgtctcatcatggaaggcgctgaatttacggaaaacattattaatggcgtcgagcgtccggttaaagccgctgaattgttcgcgtttaccttgcgtgtacgcgcaggaaacactgacgttcttactgacgcagaagaaaacgtgcgtcaaaaattacgtgcagaaggagtgatgtaatgtctaaaggtaaaaaacgttctggcgctcgccctggtcgtccgcagccgttgcgaggtactaaaggcaagcgtaaaggcgctcgtctttggtatgtaggtggtcaacaattttaattgcaggggcttcggccccttacttgaggataaattatgtctaatattcaaactggcgccgagcgtatgccgcatgacctttcccatcttggcttccttgctggtcagattggtcgtcttattaccatttcaactactccggttatcgctggcgactccttcgagatggacgccgttggcgctctccgtctttctccattgcgtcgtggccttgctattgactctactgtagacatttttactttttatgtccctcatcgtcacgtttatggtgaacagtggattaagttcatgaaggatggtgttaatgccactcctctcccgactgttaacactactggttatattgaccatgccgcttttcttggcacgattaaccctgataccaataaaatccctaagcatttgtttcagggttatttgaatatctataacaactattttaaagcgccgtggatgcctgaccgtaccgaggctaaccctaatgagcttaatcaagatgatgctcgttatggtttccgttgctgccatctcaaaaacatttggactgctccgcttcctcctgagactgagctttctcgccaaatgacgacttctaccacatctattgacattatgggtctgcaagctgcttatgctaatttgcatactgaccaagaacgtgattacttcatgcagcgttaccatgatgttatttcttcatttggaggtaaaacctcttatgacgctgacaaccgtcctttacttgtcatgcgctctaatctctgggcatctggctatgatgttgatggaactgaccaaacgtcgttaggccagttttctggtcgtgttcaacagacctataaacattctgtgccgcgtttctttgttcctgagcatggcactatgtttactcttgcgcttgttcgttttccgcctactgcgactaaagagattcagtaccttaacgctaaaggtgctttgacttataccgatattgctggcgaccctgttttgtatggcaacttgccgccgcgtgaaatttctatgaaggatgttttccgttctggtgattcgtctaagaagtttaagattgctgagggtcagtggtatcgttatgcgccttcgtatgtttctcctgcttatcaccttcttgaaggcttcccattcattcaggaaccgccttctggtgatttgcaagaacgcgtacttattcgccaccatgattatgaccagtgtttccagtccgttcagttgttgcagtggaatagtcaggttaaatttaatgtgaccgtttatcgcaatctgccgaccactcgcgattcaatcatgacttcgtgataaaagattgagtgtgaggttataacgccgaagcggtaaaaattttaatttttgccgctgaggggttgaccaagcgaagcgcggtaggttttctgcttaggagtttaatcatgtttcagacttttatttctcgccataattcaaactttttttctgataagctggttctcacttctgttactccagcttcttcggcacctgttttacagacacctaaagctacatcgtcaacgttatattttgatagtttgacggttaatgctggtaatggtggttttcttcattgcattcagatggatacatctgtcaacgccgctaatcaggttgtttctgttggtgctgatattgcttttgatgccgaccctaaattttttgcctgtttggttcgctttgagtcttcttcggttccgactaccctcccgactgcctatgatgtttatcctttggatggtcgccatgatggtggttattataccgtcaaggactgtgtgactattgacgtccttccccgtacgccgggcaataatgtttatgttggtttcatggtttggtctaactttaccgctactaaatgccgcggattggtttcgctgaatcaggttattaaagagattatttgtctccagccacttaagtgaggtgatttatgtttggtgctattgctggcggtattgcttctgctcttgctggtggcgccatgtctaaattgtttggaggcggtcaaaaagccgcctccggtggcattcaaggtgatgtgcttgctaccgataacaatactgtaggcatgggtgatgctggtattaaatctgccattcaaggctctaatgttcctaaccctgatgaggccgtccctagttttgtttctggtgctatggctaaagctggtaaaggacttcttgaaggtacgttgcaggctggcacttctgccgtttctgataagttgcttgatttggttggacttggtggcaagtctgccgctgataaaggaaaggatactcgtgattatcttgctgctgcatttcctgagcttaatgcttgggagcgtgctggtgctgatgcttcctctgctggtatggttgacgccggatttgagaatcaaaaagagcttactaaaatgcaactggacaatcagaaagagattgccgagatgcaaaatgagactcaaaaagagattgctggcattcagtcggcgacttcacgccagaatacgaaagaccaggtatatgcacaaaatgagatgcttgcttatcaacagaaggagtctactgctcgcgttgcgtctattatggaaaacaccaatctttccaagcaacagcaggtttccgagattatgcgccaaatgcttactcaagctcaaacggctggtcagtattttaccaatgaccaaatcaaagaaatgactcgcaaggttagtgctgaggttgacttagttcatcagcaaacgcagaatcagcggtatggctcttctcatattggcgctactgcaaaggatatttctaatgtcgtcactgatgctgcttctggtgtggttgatatttttcatggtattgataaagctgttgccgatacttggaacaatttctggaaagacggtaaagctgatggtattggctctaatttgtctaggaaataaccgtcaggattgacaccctcccaattgtatgttttcatgcctccaaatcttggaggcttttttatggttcgttcttattacccttctgaatgtcacgctgattattttgactttgagcgtatcgaggctcttaaacctgctattgaggcttgtggcatttctactctttctcaatccccaatgcttggcttccataagcagatggataaccgcatcaagctcttggaagagattctgtcttttcgtatgcagggcgttgagttcgataatggtgatatgtatgttgacggccataaggctgcttctgacgttcgtgatgagtttgtatctgttactgagaagttaatggatgaattggcacaatgctacaatgtgctcccccaacttgatattaataacactatagaccaccgccccgaaggggacgaaaaatggtttttagagaacgagaagacggttacgcagttttgccgcaagctggctgctgaacgccctcttaaggatattcgcgatgagtataattaccccaaaaagaaaggtattaaggatgagtgttcaagattgctggaggcctccactatgaaatcgcgtagaggctttgctattcagcgtttgatgaatgcaatgcgacaggctcatgctgatggttggtttatcgtttttgacactctcacgttggctgacgaccgattagaggcgttttatgataatcccaatgctttgcgtgactattttcgtgatattggtcgtatggttcttgctgccgagggtcgcaaggctaatgattcacacgccgactgctatcagtatttttgtgtgcctgagtatggtacagctaatggccgtcttcatttccatgcggtgcactttatgcggacacttcctacaggtagcgttgaccctaattttggtcgtcgggtacgcaatcgccgccagttaaatagcttgcaaaatacgtggccttatggttacagtatgcccatcgcagttcgctacacgcaggacgctttttcacgttctggttggttgtggcctgttgatgctaaaggtgagccgcttaaagctaccagttatatggctgttggtttctatgtggctaaatacgttaacaaaaagtcagatatggaccttgctgctaaaggtctaggagctaaagaatggaacaactcactaaaaaccaagctgtcgctacttcccaagaagctgttcagaatcagaatgagccgcaacttcgggatgaaaatgctcacaatgacaaatctgtccacggagtgcttaatccaacttaccaagctgggttacgacgcgacgccgttcaaccagatattgaagcagaacgcaaaaagagagatgagattgaggctgggaaaagttactgtagccgacgttttggcggcgcaacctgtgacgacaaatctgctcaaatttatgcgcgcttcgataaaaatgattggcgtatccaacctgca

SEQ ID NO: 2 - DNA, 32 bp, Artificial Sequence
  Staple Sequence
gttgaccacctacataccaaatacgctcctga

SEQ ID NO: 3 - DNA, 50 bp, Artificial Sequence
  Staple Sequence
cgagagtcaggtacggcggtcagtagtttgttactcgtcagaaaatcgaa

SEQ ID NO: 4 - DNA, 28 bp, Artificial Sequence
  Staple Sequence
tgcgctggtaataagacgaccaggaatt

SEQ ID NO: 5 - DNA, 40 bp, Artificial Sequence
  Staple Sequence
gttaatcgagcggcatggtcaatattaacaccaaacgtga

SEQ ID NO: 6 - DNA, 40 bp, Artificial Sequence
  Staple Sequence
tctcctcactaccatgaacaaaatgtaatccacgctcttt

SEQ ID NO: 7 - DNA, 36 bp, Artificial Sequence
  Staple Sequence
ggaagacggcagcaataaacagtagttgaaagtaca

SEQ ID NO: 8 - DNA, 32 bp, Artificial Sequence
  Staple Sequence
taaaaagtgagagcgcgaaggagtcaaggaag

SEQ ID NO: 9 - DNA, 38 bp, Artificial Sequence
  Staple Sequence
taaaatgtcaacaaaccaatccaagagaatcttccaac

SEQ ID NO: 10 - DNA, 48 bp, Artificial Sequence
  Staple Sequence
atcatcttattagggttagcctcggcatccacggcgctttgtatcagg

SEQ ID NO: 11 - DNA, 46 bp, Artificial Sequence
  Staple Sequence
gcgtcagcgaaacgcagtttttgacatatcctacaaagtccaagca

SEQ ID NO: 12 - DNA, 36 bp, Artificial Sequence
  Staple Sequence
gtggagcttgcagacccatatcgttctctaacccag

SEQ ID NO: 13 - DNA, 28 bp, Artificial Sequence
  Staple Sequence
agatgaaaccatttttgaatctctcaga

SEQ ID NO: 14 - DNA, 40 bp, Artificial Sequence
  Staple Sequence
cggtcgtcagagtgtcaaaaacgatcatcaaaagtggagg

SEQ ID NO: 15 - DNA, 46 bp, Artificial Sequence
  Staple Sequence
ataactccaaagcagcctgaaacaaagtcatttggcgagaaaagat

SEQ ID NO: 16 - DNA, 32 bp, Artificial Sequence
  Staple Sequence
ttgaacacagagggcgcggcaaaagtggtcta

SEQ ID NO: 17 - DNA, 32 bp, Artificial Sequence
  Staple Sequence
agcagccttatggccgtcaaggagcacatcgg

SEQ ID NO: 18 - DNA, 38 bp, Artificial Sequence
  Staple Sequence
cagaagcctgaatgagggaaaccataacgagcattcaa

SEQ ID NO: 19 - DNA, 50 bp, Artificial Sequence
  Staple Sequence
ttgagttgcgtagccatggcagcaaccttaatagaggccaaagcggtctg

SEQ ID NO: 20 - DNA, 40 bp, Artificial Sequence
  Staple Sequence
ttatagatatcatcttgattaagcgttaaatccaaaacgg

SEQ ID NO: 21 - DNA, 48 bp, Artificial Sequence
  Staple Sequence
gaaacgtatcggcgtgtgaatcataccctcggcagcaagacctctaat

SEQ ID NO: 22 - DNA, 38 bp, Artificial Sequence
  Staple Sequence
gactcatgatttcttaaccatactcaggcacacgaaaa

SEQ ID NO: 23 - DNA, 28 bp, Artificial Sequence
  Staple Sequence
ccaactgcgccgccaagggaagtacgtg

SEQ ID NO: 24 - DNA, 46 bp, Artificial Sequence
  Staple Sequence
tagtcatggaaccgcacgcaaagcatggtcaacgctacctgtcgac

SEQ ID NO: 25 - DNA, 40 bp, Artificial Sequence
  Staple Sequence
caatatcacaaaaatactgatagcgattgttcagtaactt

SEQ ID NO: 26 - DNA, 48 bp, Artificial Sequence
  Staple Sequence
gactcagagactcatatctaaaccgaacgtgccaagcatagaaaggtc

SEQ ID NO: 27 - DNA, 32 bp, Artificial Sequence
  Staple Sequence
gacagatttgtcattgtgagtgcgttctctac

SEQ ID NO: 28 - DNA, 36 bp, Artificial Sequence
  Staple Sequence
gacctttaactggcggcgattcgtcacaggtagcca

SEQ ID NO: 29 - DNA, 32 bp, Artificial Sequence
  Staple Sequence
atcagaaaatacgccacgcgcataacttttcc

SEQ ID NO: 30 - DNA, 50 bp, Artificial Sequence
  Staple Sequence
gacggttgacagtccccattagctgtcctattagtggttgaacagcatcg

SEQ ID NO: 31 - DNA, 40 bp, Artificial Sequence
  Staple Sequence
gcaaagtagagctgcgcaaggatacgatttaatcaagata

SEQ ID NO: 32 - DNA, 42 bp, Artificial Sequence
  Staple Sequence
aagcatgtcaatgctcagtctctttttcatcttcacactact

SEQ ID NO: 33 - DNA, 41 bp, Artificial Sequence
  Staple Sequence
ttacctccaaatgaagaataattattcatcttcacactact

SEQ ID NO: 34 - DNA, 48 bp, Artificial Sequence
  Staple Sequence
gacgggatacaattcagctcgacgtttttgactcatcttcacactact

SEQ ID NO: 35 - DNA, 48 bp, Artificial Sequence
  Staple Sequence
ggcgctgcgtaaccgtcttcctctacgcaatatcatcttcacactact

SEQ ID NO: 36 - DNA, 48 bp, Artificial Sequence
  Staple Sequence
taaggccaaagcggctaggccacagcaaggtctcatcttcacactact

SEQ ID NO: 37 - DNA, 36 bp, Artificial Sequence
  Staple Sequence
caatggcgccaggcaatggatcatcttcacactact

SEQ ID NO: 38 - DNA, 48 bp, Artificial Sequence
  Staple Sequence
ccagcgccagcgataaccggatccactgctactcatcttcacactact

SEQ ID NO: 39 - DNA, 60 bp, Artificial Sequence
  Staple Sequence
ttaagcgagcgccagaacgttttttaccgaacgtcactcaagtatcatcttcacactact

SEQ ID NO: 40 - DNA, 60 bp, Artificial Sequence
  Staple Sequence
actcgcgacagcttggtttttagtgagttttagccataacccagtcatcttcacactact

SEQ ID NO: 41 - DNA, 56 bp, Artificial Sequence
  Staple Sequence
tatcgtgtttcccgcaaggtaaacgcgagaacataaccattcatcttcacactact

SEQ ID NO: 42 - DNA, 42 bp, Artificial Sequence
  Staple Sequence
cttatcaacagggcgtaccagagtcatcatcttcacactact

SEQ ID NO: 43 - DNA, 60 bp, Artificial Sequence
  Staple Sequence
acgttccaagagctcaaagaaacgcggccatcaacactcagtaatcatcttcacactact

SEQ ID NO: 44 - DNA, 48 bp, Artificial Sequence
  Staple Sequence
agtaaatttgagcagatttgagtaattcagtgtcatcttcacactact

SEQ ID NO: 45 - DNA, 41 bp, Artificial Sequence
  Staple Sequence
ttaataatgttttccgtaaaggccatcatcttcacactact

SEQ ID NO: 46 - DNA, 56 bp, Artificial Sequence
  Staple Sequence
actttcatagccagattagagcgcatgagcaaattaaacgtcatcttcacactact

SEQ ID NO: 47 - DNA, 36 bp, Artificial Sequence
  Staple Sequence
tcgggcttcaataaactctgtcatcttcacactact

SEQ ID NO: 48 - DNA, 60 bp, Artificial Sequence
  Staple Sequence
ttaacgtatgttccatctttagcacttggtaagttggatttcgctcatcttcacactact

SEQ ID NO: 49 - DNA, 60 bp, Artificial Sequence
  Staple Sequence
gtcagttcacagaatgacacgacccagatacaaactcatcattatcatcttcacactact

SEQ ID NO: 50 - DNA, 60 bp, Artificial Sequence
  Staple Sequence
gctaaaaattaggagcctgtcgcattgcaaccaacccgatgggctcatcttcacactact

SEQ ID NO: 51 - DNA, 60 bp, Artificial Sequence
  Staple Sequence
gtcagtaatttagacagcacgtaaaggggccgaagcccctaatttcatcttcacactact

SEQ ID NO: 52 - DNA, 60 bp, Artificial Sequence
  Staple Sequence
caatagcgagggtccgccagcagtccactcgaatttagacaggctcatcttcacactact

SEQ ID NO: 53 - DNA, 36 bp, Artificial Sequence
  Staple Sequence
ccctttgtagcaatcgcgaatcatcttcacactact

SEQ ID NO: 54 - DNA, 60 bp, Artificial Sequence
  Staple Sequence
aagctagaagtcgtcgggagaggagtggaccagtagatgaagtatcatcttcacactact

SEQ ID NO: 55 - DNA, 42 bp, Artificial Sequence
  Staple Sequence
cagttgcgtaccaggaagtgcttctgtcatcttcacactact

SEQ ID NO: 56 - DNA, 41 bp, Artificial Sequence
  Staple Sequence
aaccagaacgtgaaaaagtggaagctcatcttcacactact

SEQ ID NO: 57 - DNA, 48 bp, Artificial Sequence
  Staple Sequence
gtcagtatcaagtaaacataagagagaaaacttcatcttcacactact

SEQ ID NO: 58 - DNA, 56 bp, Artificial Sequence
  Staple Sequence
gtcgcatagaaatataactggtagctttcgtattttgcgatcatcttcacactact

SEQ ID NO: 59 - DNA, 38 bp, Artificial Sequence
  Staple Sequence
cctacgagtcagtccttggggataacatcatggtgcat

SEQ ID NO: 60 - DNA, 50 bp, Artificial Sequence
  Staple Sequence
cctacgagtcagtcctcctttcaacgtgagccaggaggaagcggatgttt

SEQ ID NO: 61 - DNA, 40 bp, Artificial Sequence
  Staple Sequence
cctacgagtcagtcctgataatctggttgaacggcgaagc

SEQ ID NO: 62 - DNA, 40 bp, Artificial Sequence
  Staple Sequence
cctacgagtcagtcctactcttgtgccaattcatccacga

SEQ ID NO: 63 - DNA, 50 bp, Artificial Sequence
  Staple Sequence
cctacgagtcagtcctagtataaagaaatgccaacgcaagcctccgagca

SEQ ID NO: 64 - DNA, 50 bp, Artificial Sequence
  Staple Sequence
cctacgagtcagtcctttaatccttctcagagcgcataaagtgcaatgaa

SEQ ID NO: 65 - DNA, 60 bp, Artificial Sequence
  Staple Sequence
cctacgagtcagtcctaagttgggcatacatacaacgccctgcatacgaaaagacacgtc

SEQ ID NO: 66 - DNA, 60 bp, Artificial Sequence
  Staple Sequence
cctacgagtcagtcctcatgcggcagacgagcttagtacctcgcaacggctgcggacgac

SEQ ID NO: 67 - DNA, 40 bp, Artificial Sequence
  Staple Sequence
cctacgagtcagtcctcgactttgaatattagacatgcaa

SEQ ID NO: 68 - DNA, 60 bp, Artificial Sequence
  Staple Sequence
cctacgagtcagtccttctcttttcattttcacattctgattctgaacagcttcttaacg

SEQ ID NO: 69 - DNA, 52 bp, Artificial Sequence
  Staple Sequence
cctacgagtcagtcctactgatcagcattgggattatcataaaacatacgac

SEQ ID NO: 70 - DNA, 38 bp, Artificial Sequence
  Staple Sequence
cctacgagtcagtcctcgtcacgtcctgcgtgtagcaa

SEQ ID NO: 71 - DNA, 52 bp, Artificial Sequence
  Staple Sequence
cctacgagtcagtccttatccttatcatccttgatttcatcgctgaatagca

SEQ ID NO: 72 - DNA, 52 bp, Artificial Sequence
  Staple Sequence
cctacgagtcagtcctgatgtctcatttgaatcgttagttgatgaagccact

SEQ ID NO: 73 - DNA, 52 bp, Artificial Sequence
  Staple Sequence
cctacgagtcagtcctcaggttggtatccgaactgctttattcgtaaacaag

SEQ ID NO: 74 - DNA, 52 bp, Artificial Sequence
  Staple Sequence
cctacgagtcagtcctctgctgttaacatgcttagggattttataatagttg

SEQ ID NO: 75 - DNA, 52 bp, Artificial Sequence
  Staple Sequence
cctacgagtcagtcctgaaagacgaaaaatgtttcaccatatccttcatgaa

SEQ ID NO: 76 - DNA, 38 bp, Artificial Sequence
  Staple Sequence
cctacgagtcagtcctatagcaattcagcgcctttaag

SEQ ID NO: 77 - DNA, 58 bp, Artificial Sequence
  Staple Sequence
gtcggaagcatgcaacacagttgtggagcttgcagacccatatcgttctctaacccag

SEQ ID NO: 78 - DNA, 23 bp, Artificial Sequence
  Staple Sequence
tttctgtgttgcatgcttccgac